(19) United States
(12) Patent Application Publication
(10) Pub. No.: US 2021/0381054 A1
(43) Pub. Date: Dec. 9, 2021

(54) METHODS, SYSTEMS AND KITS FOR PREDICTING PREMATURE BIRTH CONDITION

(71) Applicant: COYOTE DIAGNOSTICS LAB (BEIJING) CO., LTD., Beijing (CN)

(72) Inventors: Xiang LI, Beijing (CN); Qubo AI, Beijing (CN)

(21) Appl. No.: 17/290,486

(22) PCT Filed: Oct. 31, 2019
(86) PCT No.: PCT/CN2019/114756

§ 371 Date: Apr. 30, 2021

(30) Foreign Application Priority Data
Oct. 31, 2018 (CN) PCT/CN2018/112965

(51) Int. Cl. C12Q 1/6883; C12Q 1/686; G16B 50/10; G16H 50/20; G16H 50/30

(52) U.S. Cl. CPC C12Q 1/6883; C12Q 1/686; G16H 50/30; G16H 50/20; G16B 50/10

(57) ABSTRACT

Methods and systems (301) are provided to predicting premature birth condition in a subject. The method for predicting in or monitoring premature birth condition in a subject comprises processing a biological sample obtained from the subject to generate data indicative of a distribution of a plurality of populations of microbes of different types in the biological sample. A presence, absence, or relative amount of an individual population of the plurality of populations of microbes may be indicative of a premature birth condition. Next, a trained algorithm may be used to process the data to determine a presence, absence, or relative amount of the individual population of microbe. Next, based on the presence, absence, or relative amount, the subject may be identified as having the premature birth condition, such as, for example, in a report.

### CLAIM OF PRIORITY

This application claims priority of PCT application PCT/CN2018/112965, filed on Oct. 31, 2018, the entire contents of which are incorporated by reference herein.

### BACKGROUND

Preterm birth is the leading cause of death among children under the age of 5 worldwide and the major cause of perinatal morbidity and mortality. In 2015, preterm birth and low birth weight accounted for about 17% of infant deaths. In the U.S., 10% of babies are born prematurely each year. One third of all premature or preterm births are caused by preterm premature rupture of membranes (PPROM). The spontaneous rupture of membranes (ROM) (i.e., the breakage of the amniotic sac) is a normal component of labor and delivery. Premature rupture of membranes (PROM) refers to the rupture of the fetal membranes prior to the onset of labor irrespective of gestational age. When PROM occurs at term, labor typically ensues spontaneously or is induced within 12 to 24 hours. Preterm premature rupture of membranes (PPROM) refers to PROM occurring prior to 37 weeks of gestation. The management of pregnancies complicated by PPROM is more challenging. PPROM complicates about 2% to 20% of all deliveries and is associated with about 18% to 20% of perinatal deaths. Management options include admission to hospital, amniocentesis to exclude intra-amniotic infection, and administration of antenatal corticosteroids and broad-spectrum antibiotics, if indicated.

The current gold standard for the diagnosis of PROM and/or PPROM includes a reviewing the patient's medical history, physical examination, and clinical assessment of pooling, nitrazine (a pH indicator dye), and/or ferning (i.e., testing for a “fern like” pattern in dry cervical mucus to check for the presence of amniotic fluid). Other diagnostic methods include identification of biomarkers, such as alpha-fetoprotein (AFP), fetal fibronectin (fFN), insulin-like growth factor binding protein 1 (IGFBP1), prolactin, beta-subunit of human chrorionic gonadotropin (I3-hCG), creatinine, urea, lactate, and placental alpha macroglobulin 1 (PAMG-1) that are present in the cervicovaginal discharge. However, such tests are conducted primarily once a potential birth condition (e.g., PPROM) occurs, but may be absent in women with intact membranes. In other words, current diagnostic tests may be unable to predict a potential premature birth such as PPROM. Early and accurate diagnosis of PROM and PPROM would allow for gestational age-specific obstetric interventions designed to optimize perinatal outcome and minimize serious complications, such as cord prolapse and infectious morbidity (e.g., chorioamnionitis and neonatal sepsis). Thus, there exists a need for rapid, accurate screening methods for premature birth that are non-invasive, cost-effective, and can be applied to pregnant women.

### SUMMARY

The present disclosure provides methods, systems, and kits for predicting premature birth condition by processing biological samples indicative of a distribution of a plurality of populations of microbes of different types. Biological samples (e.g., vaginal fluid samples) obtained from subjects may be analyzed to measure microbiome distributions. Such subjects may include subjects with premature birth condition and subjects without premature birth condition.

In an aspect, disclosed herein is method for predicting premature birth condition in a subject having an unborn baby. The method can comprise (a) processing a biological sample obtained from the subject to generate data indicative of a distribution of a plurality of populations of microbes of different types in the biological sample, wherein a presence, absence, or relative amount of an individual population of the plurality of populations of microbes is indicative of the premature birth condition in the subject; (b) using a trained algorithm to process the data indicative of the distribution of the plurality of populations of microbes to determine a presence, absence, or relative amount of the individual population of the plurality of populations of microbes in the biological sample, which trained algorithm is configured to predict the premature birth condition at an accuracy of at least 90% for independent samples; (c) based on the presence, absence, or relative amount of the individual population of the plurality of populations of microbes determined in (b), predicting the subject as having the premature birth condition in the subject at an accuracy of at least about 90%; and (d) electronically outputting a report that identifies or provides an indication of the premature birth condition in the subject.

In some embodiments, the trained algorithm can be trained with a first number of independent training samples associated with presence of a premature birth condition and a second number of independent training samples associated with absence of a premature birth condition, and the first number is no more than the second number. In some embodiments, the process (a) can comprise (i) subjecting the biological sample to conditions that are sufficient to isolate the plurality of populations of microbes, and (ii) identifying the presence, absence, or relative amount of the individual population of the plurality of populations of microbes.

In some embodiments, the plurality of populations of the plurality of populations of microbes can comprise at least 5 different populations of microbes. The at least 5 different species of microbes can comprise one or more members selected from the group consisting of Lactobacillus iners, Atopobium vagie, Escherichia coli, Prevotella bivia, Lactobacillus crispatus, Ureaplasma urealyticum, Lactobacillus gasseri, BVAB2, Enterococcus faecalis, Lactobacillus jensenii, Megasphaera 2, Mobiluncus mulieris, Staphylococcus aureus, Gardnerella vagilis, Megasphaera 1, Candida glabrata, Candida krusei, Streptococcus agalactiae, Candida albicans, Chlamydia trachomatis, Candida parapsilosis, Treponema pallidum, Mycoplasma hominis, Mobiluncus curtisii, Neisseria gonorrhoeae, Herpes simplex 1, Trichomos vagilis, Haemophilus ducreyi, Mycoplasma genitalium, Candida lusitaniae, Bacteroides fragilis, Herpes simplex 2, Candida tropicalis, and Candida dubliniensis.

In some embodiments, the method can further comprise monitoring a course of treatment for treating a premature birth condition in the subject, wherein the monitoring comprises assessing the premature birth condition in the subject at two or more time points, wherein the assessing is based at least on the presence, absence, or relative amount of the individual population of the plurality of populations of microbes determined in process (b) at each of the two or more time points.

In another aspect, disclosed herein is a computer system for predicting a premature birth condition in a subject having an unborn baby. In some embodiments, the computer system is programmed or configured to implement a method of the present disclosure, e.g. a method as set forth above. The computer system can comprise a database that is configured to store data indicative of a distribution of a plurality of populations of microbes of different types in a biological sample of the subject, wherein a presence, absence, or relative amount of an individual population of the plurality of populations of microbes is indicative of the premature birth condition in the subject; and one or more computer processors operatively coupled to the database. The one or more computer processors are individually collectively programmed to: (i) use a trained algorithm to process the data indicative of the distribution of the plurality of populations of microbes to determine a presence, absence, or relative amount of the individual population of the plurality of populations of microbes in the biological sample, which trained algorithm is configured to predict the premature birth condition at an accuracy of at least 90% for independent samples; (ii) based on the presence, absence, or relative amount of the individual population of the plurality of populations of microbes determined in (b), predict the subject as having the premature birth condition in the subject at an accuracy of at least about 90%; and (iii) electronically output a report that identifies or provides an indication of the premature birth condition in the subject.

In another aspect, disclosed herein is a non-transitory computer readable medium comprising machine-executable code that, upon execution by one or more computer processors, implements a method for predicting premature birth condition in a subject having an unborn baby. In some embodiments, the non-transitory computer readable medium comprising machine-executable code that, upon execution by one or more computer processors, implements a method of the present disclosure, e.g. a method as set forth above. The method can comprise (a) processing a biological sample obtained from the subject to generate data indicative of a distribution of a plurality of populations of microbes of different types in the biological sample, wherein a presence, absence, or relative amount of an individual population of the plurality of populations of microbes is indicative of the premature birth condition in the subject; (b) using a trained algorithm to process the data indicative of the distribution of the plurality of populations of microbes to determine a presence, absence, or relative amount of the individual population of the plurality of populations of microbes in the biological sample, which trained algorithm is configured to predict the premature birth condition at an accuracy of at least 90% for independent samples; (c) based on the presence, absence, or relative amount of the individual population of the plurality of populations of microbes determined in (b), predicting the subject as having the premature birth condition in the subject at an accuracy of at least about 90%; and (d) electronically outputting a report that identifies or provides an indication of the premature birth condition in the subject.

In another aspect, disclosed herein is a kit for predicting premature birth in a subject having an unborn baby. The kit can comprise probes for identifying a presence, absence, or relative amount of individual populations of a plurality of populations of microbes of different types in a biological sample of the subject, wherein a presence, absence, or relative amount of the individual populations of the plurality of populations of microbes in the biological is indicative of a premature birth of the subject having the unborn baby, wherein the probes are selective for the plurality of populations of microbes among other populations of microbes in the biological sample; and instructions for using the probes to process the biological sample to generate data indicative of a distribution of the plurality of populations of microbes of different types in the biological sample, to predict the premature birth at an accuracy of at least 90% for independent samples. In some embodiments, the kit is for use in a method of the present disclosure, e.g. a method as set forth above.

In another aspect, disclosed herein is the use of probes in the manufacture of a kit for the prediction of premature birth in a subject having an unborn baby. The probes is for identifying a presence, absence, or relative amount of individual populations of a plurality of populations of microbes of different types in a biological sample of said subject, wherein a presence, absence, or relative amount of said individual populations of said plurality of populations of microbes in said biological is indicative of a premature birth of said subject having said unborn baby, wherein said probes are selective for said plurality of populations of microbes among other populations of microbes in said biological sample. The prediction can comprises: (a) processing a biological sample obtained from said subject to generate data indicative of a distribution of a plurality of populations of microbes of different types in said biological sample, wherein a presence, absence, or relative amount of an individual population of said plurality of populations of microbes is indicative of said premature birth condition in said subject; (b) using a trained algorithm to process said data indicative of said distribution of said plurality of populations of microbes to determine a presence, absence, or relative amount of said individual population of said plurality of populations of microbes in said biological sample, which trained algorithm is configured to predict said premature birth condition at an accuracy of at least 90% for independent samples; (c) based on said presence, absence, or relative amount of said individual population of said plurality of populations of microbes determined in (b), predicting said subject as having said premature birth condition in said subject at an accuracy of at least about 90%; and optionally (d) electronically outputting a report that identifies or provides an indication of said premature birth condition in said subject.

In some embodiments, the kit is used in a method of the present disclosure, e.g. a method as set forth above.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example of a Receiver Operator Characteristic (ROC) curve of a Random Forest classifier configured to predict a premature birth condition based on analysis of microbe populations in vaginal samples, in accordance with some embodiments where average number of Cᵣₜ values, number of previous abortions, and age of the pregnant woman are used as variables.

FIGS. 2A-2G illustrate an example of raw assay data in accordance with embodiments of FIG. 1.

FIG. 3 illustrates an example of a Receiver Operator Characteristic (ROC) curve of a Random Forest classifier configured to predict a premature birth condition based on analysis of microbe populations in vaginal samples, in accordance with some embodiments where percentages of respective microbes, number of previous abortions, and age of the pregnant woman are used as variables.

FIGS. 4A-4F illustrate an example of raw assay data in accordance with embodiments of FIG. 3.

FIG. 5 illustrates a computer control system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an” and “the” include plural references unless the context clearly dictates otherwise. For example, the term “a cell” includes a plurality of cells, including mixtures thereof.

As used herein, the term “nucleic acid” generally refers to a polymeric form of nucleotides of any length, either deoxyribonucleotides (dNTPs) or ribonucleotides (rNTPs), or analogs thereof. Nucleic acids may have any three dimensional structure, and may perform any function, known or unknown. Non-limiting examples of nucleic acids include DNA, RNA, coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs. If present, modifications to the nucleotide structure may be made before or after assembly of the nucleic acid. The sequence of nucleotides of a nucleic acid may be interrupted by non nucleotide components. A nucleic acid may be further modified after polymerization, such as by conjugation or binding with a reporter agent.

As used herein, the terms “amplifying” and “amplification” are used interchangeably and generally refer to generating one or more copies or “amplified product” of a nucleic acid. The term “DNA amplification” generally refers to generating one or more copies of a DNA molecule or “amplified DNA product”. The term “reverse transcription amplification” generally refers to the generation of deoxyribonucleic acid (DNA) from a ribonucleic acid (RNA) template via the action of a reverse transcriptase.

As used herein, the term “target nucleic acid” generally refers to a nucleic acid molecule in a starting population of nucleic acid molecules having a nucleotide sequence whose presence, amount, and/or sequence, or changes in one or more of these, are desired to be determined. A target nucleic acid may be any type of nucleic acid, including DNA, RNA, and analogues thereof. As used herein, a “target ribonucleic acid (RNA)” generally refers to a target nucleic acid that is RNA. As used herein, a “target deoxyribonucleic acid (DNA)” generally refers to a target nucleic acid that is DNA.

As used herein, the term “subject,” generally refers to an entity or a medium that has testable or detectable genetic information. A subject can be a person or individual. A subject can be a vertebrate, such as, for example, a mammal. Non-limiting examples of mammals include murines, simians, humans, farm animals, sport animals, and pets. Other examples of subjects include food, plant, soil, and water.

As used herein, the terms “about” or “approximately,” refer to an amount that is near the stated amount by about 10%, 5%, or 1%, including increments therein. For example, “about” or “approximately” can mean a range including the particular value and ranging from 10% below that particular value and spanning to 10% above that particular value.

As used herein, the term “premature birth” generally refers to a birth that takes place more than three weeks before the baby's estimated due date. In other words, a premature birth is one that occurs before the start of the 37th week of pregnancy. A premature birth can be caused by preterm premature rupture of membranes (PPROM). In other words, the preterm premature rupture of membranes (PPROM) is one of the reasons causing a premature birth. A premature birth condition can be preterm premature rupture of membranes (PPROM). The term “premature birth” can be exchangeable with the term “premature labor”.

Biological samples (e.g., vaginal fluid samples, amniotic fluid samples) obtained from subjects may be analyzed to measure microbiome distributions, e.g., a plurality of populations of microbes of different types in the biological sample. Such subjects may include female subjects, female subjects of reproductive age, pregnant subjects, pregnant subjects with a medical history of abortions, pregnant subjects with a history of premature birth, and/or pregnant subjects with a medical history of births lacking any complications. Methods, systems, and kits are provided for predict premature birth by processing biological samples indicative of a distribution of a plurality of populations of microbes of different types. A premature birth may comprise preterm premature birth condition, preterm birth, and/or premature birth. A premature rupture of may cause chorioamnionitis, neonate sepsis, or both.

For some species of microbes, population measurements in premature birth samples (e.g., biological samples obtained from a subject that had a premature birth) may be greater than in normal samples (e.g., biological samples obtained from a subject that did not have a premature birth when giving birth). For other species of microbes, population measurements in premature birth samples (e.g., biological samples obtained from a subject that had a premature birth) may be less than in normal samples (e.g., biological samples obtained from a subject that did not have a premature birth when giving birth).

These species of microbes may be candidates for biomarkers for predicting premature birth due to their differential presence in premature birth samples versus normal biological samples. In particular, since collecting vaginal fluid samples may already be part of routine clinical examinations in pregnant women and next-generation sequencing is relatively inexpensive, microbiome distribution may be used as an early detection of premature birth (e.g., premature birth condition) as an alternative to, or in conjunction with, traditional clinical tests such as relevant biomarker identification and/or physical examination such as, but not limited to a sterile speculum exam. Microbiome distribution may be used to monitor a patient (e.g., subject who is pregnant or who is pregnant and at risk for premature birth). In such cases, the microbiome distribution of the patient may change during the monitoring phase. For example, the microbiome distribution of a patient who is at risk for premature birth may shift toward the microbiome distribution of a healthy subject (i.e., a subject that is not at risk for premature birth). Conversely, for example, the microbiome distribution of a patient who is at risk for premature birth may remain the same.

In an aspect, disclosed herein is a method for predicting a premature birth in a subject having an unborn baby. The method may comprise processing a biological sample obtained from the subject to generate data indicative of a distribution of a plurality of populations of microbes of different types in the biological sample. A presence, absence, or relative amount of an individual population of the plurality of populations of microbes may be indicative of a premature birth condition of the subject. Next, a trained algorithm may be used to process the data indicative of the distribution of the plurality of populations of microbes to determine a presence, absence, or relative amount of the individual population of the plurality of populations of microbes in the biological sample. The trained algorithm may be configured to predict the premature birth condition with an accuracy of at least about 50%, 60%, 70%, 80%, 90%, 95% or greater for at least about 10, 20, 30, 40, 50, 60, 70, 80, 90, 100, 150, 200, 250, or 300 independent samples. Next, based on the presence, absence, or relative amount of the individual population of the plurality of populations of microbes, the subject may be identified as having the premature birth condition with an accuracy of at least about 50%, 60%, 70%, 80%, 90%, 95% or greater. A report may then be electronically outputted that identifies or provides an indication of the premature birth condition in the subject. The method can be performed at different time during the pregnancy of the subject, such that a progression or regression of the premature birth condition can be obtained.

### Processing Biological Samples

The biological samples may comprise vaginal fluid samples from a human subject. The vaginal fluid samples may be stored in a variety of storage conditions before processing, such as different temperatures (e.g., at room temperature, under refrigeration or freezer conditions, at 4° C., at −18° C., −20° C., or at −80° C.) or different preservatives (e.g., alcohol, formaldehyde, or potassium dichromate). The biological samples may comprise another source of vaginal microbiome from a human subject, such as an amniotic fluid sample. In some cases, the amniotic fluid sample may be obtained when performing amniocentesis.

The biological sample may be obtained from a subject with a disease or disorder, from a subject that is suspected of having the disease or disorder, or from a subject that does not have or is not suspected of having the disease or disorder. The disease or disorder may be a premature birth condition, a preterm premature birth condition, an abortion, a preterm birth, a gestational diabetes, a preeclampsia, a miscarriage, a hypertension, a premature delivery, an umbilical cord prolapse, an umbilical cord compression, an amniotic fluid embolism, a uterine bleeding, a placenta previa, a placental abruption, a placenta accreta, a placental insufficiency, an infectious disease, an immune disorder or disease, a cancer, a genetic disease, a degenerative disease, a lifestyle disease, an injury, a rare disease, and/or an age related disease. The infectious disease may be caused by bacteria, viruses, fungi and/or parasites. The cancer may be a uterine cancer, an endometrial cancer, a cervical cancer, or an ovarian cancer. The sample may be taken before and/or after treatment of a subject with a disease or disorder. Samples may be taken before and/or after the disease and disorder occurs. Samples may be taken during a treatment or a treatment regime. Multiple samples may be taken from a subject to monitor the effects of the treatment over time. Samples may be taken during a pregnancy. Multiple samples may be taken from a pregnant subject to monitor the fetus and/or placental membrane development over time. The sample may be taken from a subject known or suspected of having a premature birth condition for which a definitive positive or negative diagnosis is not available via clinical tests such as a pooling test, a nitrazine test, a fern test, and/or a fibronectin and alpha-fetoprotein test.

The sample may be taken from a subject suspected of having a disease or a disorder. The sample may be taken from a subject experiencing symptoms such as leakage of amniotic fluid from the vagina. The sample may be taken from a subject having explained symptoms. The sample may be taken from a subject at risk of developing a disease or disorder due to factors such as medical history, age, environmental exposure, lifestyle risk factors, or presence of other known risk factors. Non-limiting examples of risk factors for PROM include infections, cigarette smoking during pregnancy, illicit drug use during pregnancy, having had PROM and/or a preterm delivery in previous pregnancies, polyhydramnios, multiple gestation, bleeding anytime during the pregnancy, invasive procedures such as amniocentesis, nutritional deficits, cervical insufficiency, low socioeconomic status, and being underweight. The infections that may be risk factors for PROM include urinary tract infections, sexually transmitted diseases, lower genital infections such as bacterial vaginosis, and infections within the amniotic sac membranes.

After obtaining a biological sample from the subject, the biological sample obtained from the subject may be processed to generate data indicative of a distribution of a plurality of populations of microbes of different types in the biological sample. A presence, absence, or relative amount of an individual population of the plurality of populations of microbes may be indicative of a premature birth condition such as a premature birth condition. Processing the biological sample obtained from the subject may comprise (i) subjecting the biological sample to conditions that are sufficient to isolate the plurality of populations of microbes, and (ii) identifying the presence, absence, or relative amount of the individual population of the plurality of populations of microbes.

The plurality of populations of microbes may be isolated by extracting nucleic acid molecules from the biological sample, and subjecting the nucleic acid molecules to sequencing to identify the presence, absence, or relative amount of the individual populations of microbes of the plurality of populations of microbes. The nucleic acid molecules may comprise deoxyribonucleic acid (DNA) or ribonucleic acid (RNA). The nucleic acid molecules may comprise DNA or RNA molecules of one or more microbial populations. The nucleic acid molecules (e.g., DNA or RNA) may be extracted from the biological sample by a variety of methods, such as a FastDNA Kit protocol from MP Biomedicals, a QIAamp DNA stool mini kit from Qiagen, or a stool DNA isolation kit protocol from Norgen Biotek. The extraction method may extract all DNA molecules from a sample. Alternatively, the extract method may selectively extract a portion of DNA molecules from a sample, e.g., by targeting certain genes such as 16S ribosomal RNA (rRNA) of one or more microbial species in the DNA molecules. Extracted RNA molecules from a sample may be converted to DNA molecules by reverse transcription (RT).

The sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, semiconductor sequencing, pyrosequencing, sequencing-by-synthesis (SBS), sequencing-by-ligation, and sequencing-by-hybridization, RNA-Seq (Illumina).

The sequencing may comprise nucleic acid amplification (e.g., of DNA or RNA molecules). In some embodiments, the nucleic acid amplification is polymerase chain reaction (PCR). A suitable number of rounds of PCR (e.g., PCR, qPCR, reverse-transcriptase PCR, digital PCR, etc.) may be performed to sufficiently amplify an initial amount of nucleic acid (e.g., DNA) to a desired input quantity for subsequent sequencing. In some cases, the PCR may be used for global amplification of nucleic acids. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. The PCR may comprise targeted amplification of one or more genomic loci, such as genomic loci corresponding to one or more 16S ribosomal RNA (rRNA) genes.

The sequencing may comprise use of simultaneous reverse transcription (RT) and polymerase chain reaction (PCR), such as a OneStep RT-PCR kit protocol by Qiagen, NEB, Thermo Fisher Scientific, or Bio-Rad.

DNA or RNA molecules may be tagged, e.g., with identifiable tags, to allow for multiplexing of a plurality of samples. Any number of DNA or RNA samples may be multiplexed. For example a multiplexed reaction may contain DNA or RNA from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial samples. For example, a plurality of samples may be tagged with sample barcodes such that each DNA molecule may be traced back to the sample (and the subject) from which the DNA molecule originated. Such tags may be attached to DNA or RNA molecules by ligation or by PCR amplification with primers.

After subjecting the nucleic acid molecules to sequencing, suitable bioinformatics processes may be performed on the sequence reads to generate the data indicative of a distribution of a plurality of populations of microbes of different types in the biological sample. For example, the sequence reads may be aligned to one or more reference genomes (e.g., a genome of one or more bacterial species). The aligned sequence reads may be quantified at one or more genomic loci to generate the data indicative of a distribution of a plurality of populations of microbes of different types in the biological sample. For example, quantification of sequences corresponding to a plurality of conserved and/or non-conserved genomic loci may generate data indicative of a distribution of a plurality of populations of microbes of different types in the biological sample. Quantification of sequences may be expressed as, or converted to, units of operational taxonomic units (OTUs) for one or more microbial populations. The OTU measurements may comprise un-normalized or normalized values. The OTUs may be measured at the microbial (e.g., bacterial) genus level or the microbial species level. A collection of OTU data corresponding to a plurality of bacterial genera and/or species in a biological sample may be indicative of a distribution of a plurality of populations of microbes of different types in the biological sample. A presence, absence, or relative amount of individual populations of microbes of the plurality of populations of microbes may be inferred from the collection of OTU data. This presence, absence, or relative amount of individual populations of microbes of the plurality of populations of microbes inferred from the collection of OTU data may be indicative of a distribution of a plurality of populations of microbes of different types in the biological sample.

The premature birth condition may be identified or a progression or regression of the premature birth condition (e.g., PPROM) may be monitored in the subject by using probes configured to selectively enrich nucleic acid (e.g., DNA or RNA) molecules corresponding to the individual populations of microbes. The probes may be nucleic acid primers. The probes may have sequence complementarity with nucleic acid sequences from one or more of the individual populations of microbes.

The plurality of populations of microbes may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 or greater different populations of microbes. The plurality of populations of microbes may comprise different species of microbes. The plurality of populations of microbes may comprise one or more members selected from the group consisting of Lactobacillus iners, Atopobium vagie, Escherichia coli, Prevotella bivia, Lactobacillus crispatus, Ureaplasma urealyticum, Lactobacillus gasseri, BVAB2, Enterococcus faecalis, Lactobacillus jensenii, Megasphaera 2, Mobiluncus mulieris, Staphylococcus aureus, Gardnerella vagilis, Megasphaera 1, Candida glabrata, Candida krusei, Streptococcus agalactiae, Candida albicans, Chlamydia trachomatis, Candida parapsilosis, Treponema pallidum, Mycoplasma hominis, Mobiluncus curtisii, Neisseria gonorrhoeae, Herpes simplex 1, Trichomos vagilis, Haemophilus ducreyi, Mycoplasma genitalium, Candida lusitaniae, Bacteroides fragilis, Herpes simplex 2, Candida tropicalis, and Candida dubliniensis. The plurality of populations of microbes may comprise one or more members selected from the group consisting of Lactobacillus, Escherichia, Prevotella, Enterococcus, Candida, Staphylococcus, and Herpes.

The biological sample may be processed to identify a distribution of a plurality of populations of microbes in the biological sample without any nucleic acid extraction. For example, the processing may comprise assaying the biological sample using probes that are selected for the plurality of populations of microbes. The plurality of populations of microbes may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 or greater different populations of microbes. The plurality of populations of microbes may comprise different species of microbes. The plurality of populations of microbes may comprise one or more members selected from the group consisting of Lactobacillus iners, Atopobium vagie, Escherichia coli, Prevotella bivia, Lactobacillus crispatus, Ureaplasma urealyticum, Lactobacillus gasseri, BVAB2, Enterococcus faecalis, Lactobacillus jensenii, Megasphaera 2, Mobiluncus mulieris, Staphylococcus aureus, Gardnerella vagilis, Megasphaera 1, Candida glabrata, Candida krusei, Streptococcus agalactiae, Candida albicans, Chlamydia trachomatis, Candida parapsilosis, Treponema pallidum, Mycoplasma hominis, Mobiluncus curtisii, Neisseria gonorrhoeae, Herpes simplex 1, Trichomos vagilis, Haemophilus ducreyi, Mycoplasma genitalium, Candida lusitaniae, Bacteroides fragilis, Herpes simplex 2, Candida tropicalis, and Candida dubliniensis. The plurality of populations of microbes comprise one or more members selected from the group consisting of Lactobacillus gasseri, Gardnerella vagilis, Atopobium vagie, Ureaplasma urealyticum and Lactobacillus iners.

The probes may be nucleic acid molecules (e.g., DNA or RNA) having sequence complementarity with nucleic acid sequences (e.g., DNA or RNA) of the plurality of populations of microbes. These nucleic acid molecules may be primers or enrichment sequences. The assaying of the biological sample using probes that are selected for the plurality of populations of microbes may comprise use of array hybridization, polymerase chain reaction (PCR), or nucleic acid sequencing (e.g., DNA sequencing or RNA sequencing).

The processing may comprise assaying the biological sample using probes that are selective for the plurality of populations of microbes among other populations of microbes in the biological sample. These probes may be nucleic acid molecules (e.g., DNA or RNA) having sequence complementarity with nucleic acid sequences (e.g., DNA or RNA) of the plurality of populations of microbes. These nucleic acid molecules may be primers or enrichment sequences. The assaying may comprise use of array hybridization, polymerase chain reaction (PCR), or nucleic acid sequencing (e.g., DNA sequencing or RNA sequencing).

The assay readouts may be quantified at one or more genomic loci to generate the data indicative of a distribution of a plurality of populations of microbes of different types in the biological sample. For example, quantification of array hybridization or polymerase chain reaction (PCR) corresponding to a plurality of conserved and/or non-conserved genomic loci may generate data indicative of a distribution of a plurality of populations of microbes of different types in the biological sample. Assay readouts may comprise quantitative PCR (qPCR) values, digital PCR (dPCR) values, digital droplet PCR (ddPCR) values, fluorescence values, etc. Quantification of array hybridization or polymerase chain reaction (PCR) may be expressed as, or converted to, units of operational taxonomic units (OTUs) for one or more microbial populations. The OTU measurements may comprise un-normalized or normalized values. The OTUs may be measured at the microbial (e.g., bacterial) genus level or the microbial species level. A collection of OTU data corresponding to a plurality of bacterial genera and/or species in a biological sample may be indicative of a distribution of a plurality of populations of microbes of different types in the biological sample. A presence, absence, or relative amount of individual populations of microbes of the plurality of populations of microbes may be inferred from the collection of OTU data. This presence, absence, or relative amount of individual populations of microbes of the plurality of populations of microbes inferred from the collection of OTU data may be indicative of a distribution of a plurality of populations of microbes of different types in the biological sample.

### Kits

Provided herein are kits for predicting or predicting a premature birth condition in a pregnant subject. A kit may comprise probes for identifying a presence, absence, or relative amount of individual population of a plurality of populations of microbes of different types in a biological sample of the subject. A presence, absence, or relative amount of the individual population of the plurality of populations of microbes in the biological may be indicative of a premature birth condition. The probes may be selective for the plurality of populations of microbes among other populations of microbes in the biological sample. A kit may comprise instructions for using the probes to process the biological sample to generate data indicative of a distribution of the plurality of populations of microbes of different types in the biological sample.

The probes in the kit may be selective for the plurality of populations of microbes among other populations of microbes in the biological sample. The probes in the kit may be configured to selectively enrich nucleic acid (e.g., DNA or RNA) molecules corresponding to the individual populations of microbes. The probes in the kit may be nucleic acid primers. The probes in the kit may have sequence complementarity with nucleic acid sequences from one or more of the individual populations of microbes. The plurality of populations of microbes may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 or greater different populations of microbes. The plurality of populations of microbes may comprise different species of microbes. The plurality of populations of microbes may comprise one or more members selected from the group consisting of Lactobacillus iners, Atopobium vagie, Escherichia coli, Prevotella bivia, Lactobacillus crispatus, Ureaplasma urealyticum, Lactobacillus gasseri, BVAB2, Enterococcus faecalis, Lactobacillus jensenii, Megasphaera 2, Mobiluncus mulieris, Staphylococcus aureus, Gardnerella vagilis, Megasphaera 1, Candida glabrata, Candida krusei, Streptococcus agalactiae, Candida albicans, Chlamydia trachomatis, Candida parapsilosis, Treponema pallidum, Mycoplasma hominis, Mobiluncus curtisii, Neisseria gonorrhoeae, Herpes simplex 1, Trichomos vagilis, Haemophilus ducreyi, Mycoplasma genitalium, Candida lusitaniae, Bacteroides fragilis, Herpes simplex 2, Candida tropicalis, and Candida dubliniensis. The plurality of populations of microbes may comprise one or more members selected from the group consisting of Lactobacillus gasseri, Gardnerella vagilis, Atopobium vagie, Ureaplasma urealyticum and Lactobacillus iners.

The instructions in the kit may comprise instructions to assay the biological sample using the probes that are selective for the plurality of populations of microbes among other populations of microbes in the biological sample. These probes may be nucleic acid molecules (e.g., DNA or RNA) having sequence complementarity with nucleic acid sequences (e.g., DNA or RNA) of the plurality of populations of microbes. These nucleic acid molecules may be primers or enrichment sequences. The instructions to assay the biological sample may comprise introductions to perform array hybridization, polymerase chain reaction (PCR), or nucleic acid sequencing (e.g., DNA sequencing or RNA sequencing) to process the biological sample to generate data indicative of a distribution of a plurality of populations of microbes of different types in the biological sample. A presence, absence, or relative amount of individual populations of microbes of the plurality of populations of microbes may be indicative of a premature birth condition.

The instructions in the kit may comprise instructions to measure and interpret assay readouts, which may be quantified at one or more genomic loci to generate the data indicative of a distribution of a plurality of populations of microbes of different types in the biological sample. For example, quantification of array hybridization or polymerase chain reaction (PCR) corresponding to a plurality of conserved and/or non-conserved genomic loci may generate data indicative of a distribution of a plurality of populations of microbes of different types in the biological sample. Assay readouts may comprise quantitative PCR (qPCR) values, digital PCR (dPCR) values, digital droplet PCR (ddPCR) values, fluorescence values, etc. Quantification of array hybridization or polymerase chain reaction (PCR) may be expressed as, or converted to, units of operational taxonomic units (OTUs) for one or more microbial populations. The OTU measurements may comprise un-normalized or normalized values. The OTUs may be measured at the microbial (e.g., bacterial) genus level or the microbial species level. A collection of OTU data corresponding to a plurality of bacterial genera and/or species in a biological sample may be indicative of a distribution of a plurality of populations of microbes of different types in the biological sample. A presence, absence, or relative amount of individual populations of microbes of the plurality of populations of microbes may be inferred from the collection of OTU data. This presence, absence, or relative amount of individual populations of microbes of the plurality of populations of microbes inferred from the collection of OTU data may be indicative of a distribution of a plurality of populations of microbes of different types in the biological sample.

### Trained Algorithms

After processing a biological sample from the subject, a trained algorithm may be used to process the data indicative of the distribution of the plurality of populations of microbes (e.g., microbiome data) to determine a presence, absence, or relative amount of the individual population of the plurality of populations of microbes in the biological sample. In some embodiments, the trained algorithm may be configured to identify or predict a premature birth condition with an accuracy of at least 86.67% for independent samples. In some embodiments, the trained algorithm may be configured to identify or predict a premature birth condition with an accuracy of at least 93.33%. The accuracy may be increased with more sample data being available for training the algorithm.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network, or a deep learning algorithm. The trained algorithm may comprise an unsupervised machine learning algorithm.

The trained algorithm may be configured to accept a plurality of input variables and to produce one or more output values based on the plurality of input variables. The plurality of input variables may comprise data indicative of the distribution of the plurality of populations of microbes (e.g., microbiome data). For example, an input variable may comprise data indicative of a distribution of a population of microbes (e.g., a bacterial genus or bacterial species) in a subject's vaginal sample.

In addition to the microbiome data, other factors such as relevant basic personal information and clinical information of the subjects can be used as input variables to train the algorithm. In some embodiments, the basic personal information of the subjects comprise one or more of the age, gestational weeks and the like. In some embodiments, the clinical information of the subjects include one or more of the medical history of abortion, the medical history of diseases and the like.

The trained algorithm may comprise a classifier, such that each of the one or more output values comprises one of a fixed number of possible values (e.g., a linear classifier, a logistic regression classifier, etc.) indicating a classification of the biological sample by the classifier. The trained algorithm may comprise a binary classifier, such that each of the one or more output values comprises one of two values (e.g., {0, 1}, {positive, negative}, or {premature birth, non-premature birth}) indicating a classification of the biological sample by the classifier. The trained algorithm may be another type of classifier, such that each of the one or more output values comprises one of more than two values (e.g., {0, 1, 2}, {positive, negative, or indeterminate}, or {premature birth, non-premature birth, or indeterminate}) indicating a classification of the biological sample by the classifier. The output values may comprise descriptive labels, numerical values, or a combination thereof. Some of the output values may comprise descriptive labels. Such descriptive labels may provide an identification or indication of the disease or disorder state of the subject, and may comprise, for example, positive, negative, premature birth, non-premature birth, or indeterminate. Such descriptive labels may provide an identification of a treatment for the subject's disease or disorder state, and may comprise, for example, a therapeutic intervention, a duration of the therapeutic intervention, and/or a dosage of the therapeutic intervention. Such descriptive labels may provide an identification of secondary clinical tests that may be appropriate to perform on the subject, and may comprise, for example, a blood test, an ultrasound scan, a fern test, an indigo carmine dye test, an immune-chromatological test, a nitrazine test, a pooling test, detection of cervical length by B-ultrasound, Elisa detection of fetal protein, and/or detection of 7 maternal plasma proteins with Elisa or protein chip. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a prediction of the course of treatment to treat the disease or disorder state of the subject and may comprise, for example, an indication of an expected duration of efficacy of the course of treatment. Some numerical values may be mapped to descriptive labels, for example, by mapping 1 to “positive” and 0 to “negative”.

Some of the output values may be assigned based on one or more cutoff values. For example, a binary classification of samples may assign an output value of “positive” or 1 if the sample indicates that the subject has at least a 50% probability of having a premature birth. For example, a binary classification of samples may assign an output value of “negative” or 0 if the sample indicates that the subject has less than a 50% probability of having a premature birth. In this case, a single cutoff value of 50% is used to classify samples into one of the two possible binary output values. Examples of single cutoff values may include 1%, 2%, 5%, 10%, 15%, 20%, 25%, 30%, 35%, 40%, 45%, 50%, 55%, 60%, 65%, 70%, 75%, 80%, 85%, 90%, 95%, 98%, and 99%.

As another example, a classification of samples may assign an output value of “positive” or 1 if the sample indicates that the subject has a probability of having a premature birth of at least 50%, at least 55%, at least 60%, at least 65%, at least 70%, at least 75%, at least 80%, at least 85%, at least 90%, at least 95%, at least 98%, or at least 99%. The classification of samples may assign an output value of “positive” or 1 if the sample indicates that the subject has a probability of having a premature birth of more than 50%, more than 55%, more than 60%, more than 65%, more than 70%, more than 75%, more than 80%, more than 85%, more than 90%, more than 95%, more than 98%, or more than 99%. The classification of samples may assign an output value of “negative” or 0 if the sample indicates that the subject has a probability of having a premature birth of less than 50%, less than 45%, less than 40%, less than 35%, less than 30%, less than 25%, less than 20%, less than 10%, less than 5%, less than 2%, or less than 1%. The classification of samples may assign an output value of “negative” or 0 if the sample indicates that the subject has a probability of having a premature birth of no more than 50%, no more than 45%, no more than 40%, no more than 35%, no more than 30%, no more than 25%, no more than 20%, no more than 10%, no more than 5%, no more than 2%, or no more than 1%. The classification of samples may assign an output value of “indeterminate” or 2 if the sample has not been classified as “positive”, “negative”, 1, or 0. In this case, a set of two cutoff values is used to classify samples into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify samples into one of n+1 possible output values, where n is any positive integer.

The trained algorithm may be trained with a plurality of independent training samples. Each of the independent training samples may comprise a biological sample from a subject, associated data obtained by processing the biological sample (as described elsewhere herein), and one or more known output values corresponding to the biological sample (e.g., a premature birth, or a full term pregnancy delivery). Independent training samples may comprise biological samples and associated data and outputs obtained from a plurality of different subjects. Independent training samples may be associated with presence of the premature birth (e.g., training samples comprising biological samples and associated data and outputs obtained from a plurality of subjects known to have the premature birth). Independent training samples may be associated with absence of the premature birth (e.g., training samples comprising biological samples and associated data and outputs obtained from a plurality of subjects who are known to not have the premature birth).

The trained algorithm may be trained with at least 20, at least 40, at least 50, at least 100, at least 150, at least 200, at least 250, at least 300, at least 350, at least 400, at least 450, or at least 500 independent training samples. The independent training samples may comprise samples associated with presence of the premature birth condition and/or samples associated with absence of the premature birth condition. The trained algorithm is trained with no more than 500, no more than 450, no more than 400, no more than 350, no more than 300, no more than 250, no more than 200, no more than 150, no more than 100, no more than 50, or no more than 20 independent training samples associated with presence of the premature birth condition. In some embodiments, the biological sample is independent of samples used to train the trained algorithm.

The trained algorithm may be trained with a first number of independent training samples associated with presence of the premature birth condition and a second number of independent training samples associated with absence of the premature birth condition. The first number of independent training samples associated with presence of the premature birth condition may be no more than the second number of independent training samples associated with absence of the premature birth condition. The first number of independent training samples associated with presence of the premature birth condition may be equal to the second number of independent training samples associated with absence of the premature birth condition. The first number of independent training samples associated with presence of the premature birth condition may be greater than the second number of independent training samples associated with absence of the premature birth condition.

The trained algorithm may be configured to predict the premature birth condition with an accuracy of at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% for independent samples. In an embodiment, the trained algorithm may be configured to predict the premature birth condition with an accuracy of at least 86.67%. In another embodiment, the trained algorithm may be configured to predict the premature birth condition with an accuracy of at least 93.33%. The accuracy of predicting the premature birth condition by the trained algorithm may be calculated as the proportion of (1) independent test samples that are correctly predicted as having the premature birth condition and (2) independent test samples that are correctly predicted as not having the premature birth condition among all independent test samples.

The trained algorithm may be configured to predict the premature birth condition with a sensitivity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% for at least 100 independent samples. In an embodiment, the trained algorithm may be configured to predict the premature birth condition with a sensitivity of at least 83.33%. The sensitivity of predicting the premature birth condition by the trained algorithm may be calculated as the proportion of independent test samples that are correctly predicted as having the premature birth condition among a sum of (1) independent test samples that are correctly predicted as having the premature birth condition and (2) independent test samples that are incorrectly predicted as not having the premature birth condition.

The trained algorithm may be configured to predict the premature birth condition with a specificity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% for at least 100 independent samples. In an embodiment, the trained algorithm may be configured to predict the premature birth condition with a specificity of at least 88.89%. In another embodiment, the trained algorithm may be configured to predict the premature birth condition with a specificity of 100%. The specificity of predicting the premature birth condition by the trained algorithm may be calculated as the proportion of independent test samples that are correctly predicted as not having the premature birth condition among a sum of (1) independent test samples that are correctly predicted as not having the premature birth condition and (2) independent test samples that are incorrectly predicted as having the premature birth condition.

The trained algorithm may be configured to predict the premature birth condition with a positive predictive value (PPV) of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% for at least 100 independent samples. In an embodiment, the trained algorithm may be configured to predict the premature birth condition with a PPV of 83.33%. In another embodiment, the trained algorithm may be configured to predict the premature birth condition with a PPV of 100%. The PPV of predicting the premature birth condition by the trained algorithm may be calculated as the proportion of independent test samples that are correctly predicted as having the premature birth condition among a sum of (1) independent test samples that are correctly predicted as having the premature birth condition and (2) independent test samples that are incorrectly predicted as having the premature birth condition. A PPV may also be referred to as a precision.

The trained algorithm may be configured to predict the premature birth condition with an F-score of at least about 0.05, at least about 0.10, at least about 0.15, at least about 0.20, at least about 0.25, at least about 0.30, at least about 0.35, at least about 0.40, at least about 0.50, at least about 0.65, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99. In an embodiment, the trained algorithm may be configured to predict the premature birth condition with an F-score of 0.8333. In another embodiment, the trained algorithm may be configured to predict the premature birth condition with an F-score of 0.9091%. The F-score of predicting the premature birth condition by the trained algorithm may be calculated as the harmonic mean of the precision and the recall of the identification.

The trained algorithm may be configured to predict the premature birth condition with an Area-Under-Curve (AUC) of at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99. In an embodiment, the trained algorithm may be configured to predict the premature birth condition with a AUC of 94.44%. In another embodiment, the trained algorithm may be configured to predict the premature birth condition with a AUC of 98.15%. The AUC may be calculated as an integral of the Receiver Operator Characteristic (ROC) curve (e.g., the area under the ROC curve) associated with the trained algorithm in predicting biological samples as having or not having the premature birth condition.

The trained algorithm may be adjusted or tuned to improve the accuracy, PPV, sensitivity, specificity, AUC or F-score of predicting the premature birth condition. The trained algorithm may be adjusted or tuned by adjusting parameters of the trained algorithm (e.g., a set of cutoff values used to classify a sample as described elsewhere herein, or weights of a neural network). The trained algorithm may be adjusted or tuned continuously during the training process or after the training process has completed.

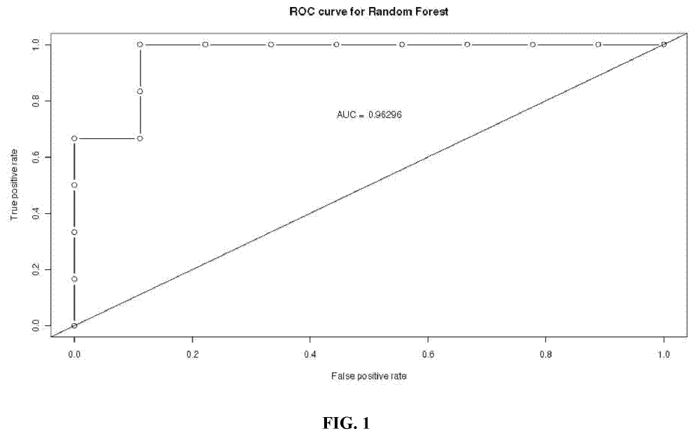

FIG. 1 illustrates an example of a Receiver Operator Characteristic (ROC) curve of a Random Forest (RF) classifier configured to predict premature birth condition based on analysis of microbe populations in vaginal samples, in accordance with some embodiments. In this example, the age of the subject, medical history of an abortion of the subject, and average Cᵣₜ values (i.e., relative threshold cycle of PCR amplification curve) were used as variables to train the algorithm.

The trained algorithm comprised a Random Forest classifier for predicting premature birth condition, which was trained by performing a plurality of successive runs. For each of the plurality of successive runs, a training partition was performed, in which at least 200, 250 or 300 biological samples were randomly selected as the training set (e.g., a set of independent training samples) for the Random Forest algorithm, and at least 20 biological samples (e.g., which was not previously selected for the training set) were designated as the testing set (e.g., a set of independent test samples). In an example, 44 biological samples were used as testing set.

The average performance metrics of this Random Forest classifier were:

Mean sensitivity ˜83.33%
Mean specificity ˜88.89%
Mean accuracy ˜86.67%
Mean precision ˜83.33%

### Mean F-Score ˜0.8333

### Mean Area Under ROC Curve (AUC) ˜0.963

As further verification of the effectiveness of the Random Forest classifier, a blind-test data set were inputted into this trained Random Forest classifier, and a prediction accuracy of 86.67% was observed. In particular, after careful tuning of the probability cutoff value based on the F-Score curve (e.g., by adjusting the probability cutoff value to increase the F-Score value as close to 1 as possible), an even higher accuracy can be achieved for this blind-test data.

In an example, the blind-test data set can comprise 44 samples, and the age of the subject, medical history of an abortion of the subject, and average Cᵣₜ values were used as variables to train the algorithm. The data of 44 test samples, including the predicted probability of premature birth condition (PBC) and predicted probability of normal birth (NORMAL) based on analysis of microbe populations in vaginal samples as well as actual birth result of each test sample, are shown in Table 1.

TABLE 1

| | Predicted | Predicted | | |
|---|---|---|---|---|
| | probability | probability | Predicted | Actual |
| Testing sample | of NORMAL | of Premature | birth | birth |
| ID | (CRT) | Birth | result | result |
| 101002000481 | 13.6% | 86.4% | PROM | PROM |
| 101002000154 | 50.4% | 49.6% | NORMAL | PROM |
| 101002000274 | 40.0% | 60.0% | PROM | PROM |
| 101002000371 | 24.8% | 75.2% | PROM | PROM |
| 101002000077 | 30.8% | 69.2% | PROM | PROM |
| 101002000151 | 25.0% | 75.0% | PROM | PROM |
| 101002000156 | 31.8% | 68.2% | PROM | PROM |
| 101002000265 | 27.0% | 73.0% | PROM | PROM |
| 101002000324 | 36.0% | 64.0% | PROM | PROM |
| 101002000333 | 25.0% | 75.0% | PROM | PROM |
| 101002000345 | 22.0% | 78.0% | PROM | PROM |
| 101002000352 | 36.6% | 63.4% | PROM | PROM |
| 101002000380 | 37.0% | 63.0% | PROM | PROM |
| 101002000390 | 45.6% | 54.4% | PROM | PROM |
| 101002000334 | 49.0% | 51.0% | PROM | PROM |
| 101002000266 | 22.2% | 77.8% | PROM | PROM |
| 101002000279 | 41.0% | 59.0% | PROM | PROM |
| 101002000373 | 22.6% | 77.4% | PROM | PROM |
| 101002000075 | 85.0% | 15.0% | NORMAL | NORMAL |
| 101002000078 | 94.4% | 5.6% | NORMAL | NORMAL |
| 101002000106 | 93.6% | 6.4% | NORMAL | NORMAL |
| 101002000109 | 80.0% | 20.0% | NORMAL | NORMAL |
| 101002000128 | 91.4% | 8.6% | NORMAL | NORMAL |
| 101002000130 | 83.0% | 17.0% | NORMAL | NORMAL |
| 101002000138 | 61.6% | 38.4% | NORMAL | NORMAL |
| 101002000157 | 76.0% | 24.0% | NORMAL | NORMAL |
| 101002000163 | 65.2% | 34.8% | NORMAL | NORMAL |
| 101002000264 | 86.6% | 13.4% | NORMAL | NORMAL |
| 101002000270 | 56.8% | 43.2% | NORMAL | NORMAL |
| 101002000271 | 66.0% | 34.0% | NORMAL | NORMAL |
| 101002000272 | 67.4% | 32.6% | NORMAL | NORMAL |
| 101002000278 | 85.0% | 15.0% | NORMAL | NORMAL |
| 101002000286 | 94.0% | 6.0% | NORMAL | NORMAL |
| 101002000295 | 73.6% | 26.4% | NORMAL | NORMAL |
| 101002000312 | 67.8% | 32.2% | NORMAL | NORMAL |
| 101002000316 | 47.6% | 52.4% | PROM | NORMAL |
| 101002000317 | 83.4% | 16.6% | NORMAL | NORMAL |
| 101002000325 | 83.6% | 16.4% | NORMAL | NORMAL |
| 101002000329 | 78.4% | 21.6% | NORMAL | NORMAL |
| 101002000370 | 87.0% | 13.0% | NORMAL | NORMAL |
| 101002000374 | 87.2% | 12.8% | NORMAL | NORMAL |
| 101002000381 | 96.0% | 4.0% | NORMAL | NORMAL |
| 101002000384 | 84.2% | 15.8% | NORMAL | NORMAL |
| 101002000440 | 82.2% | 17.8% | NORMAL | NORMAL |

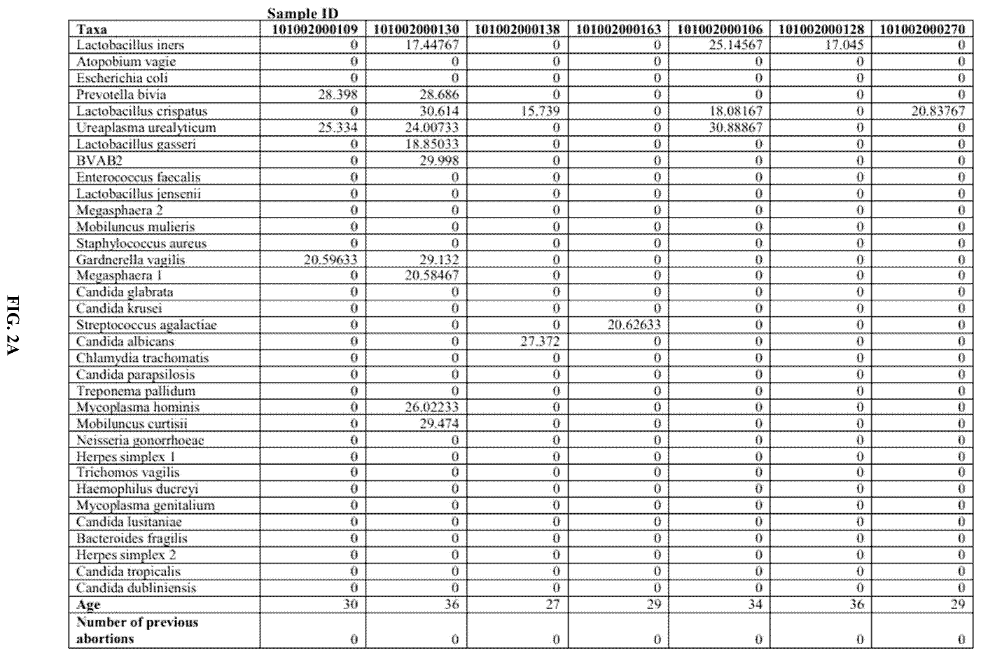

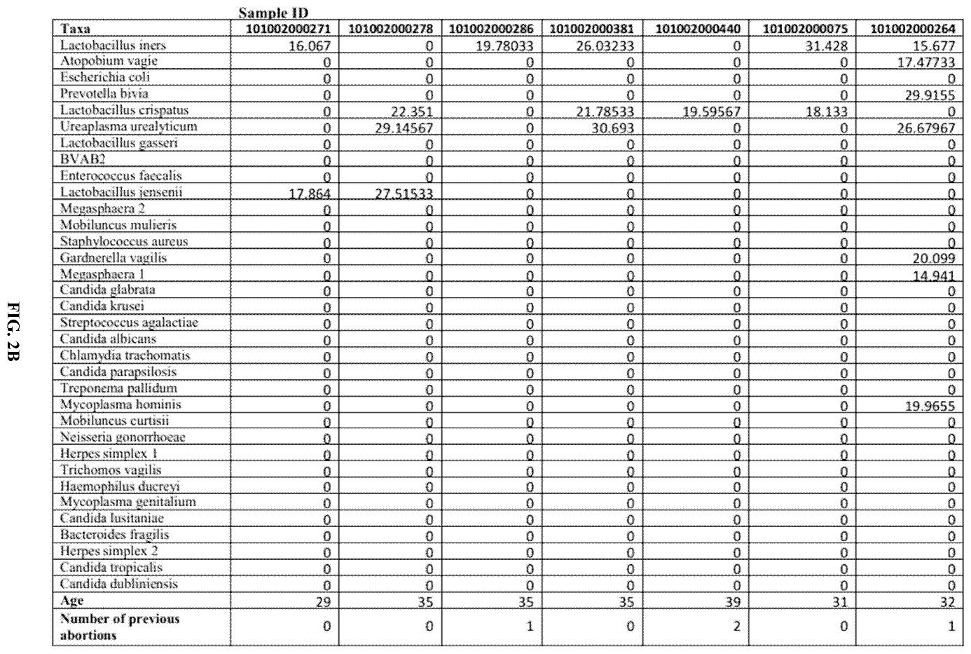

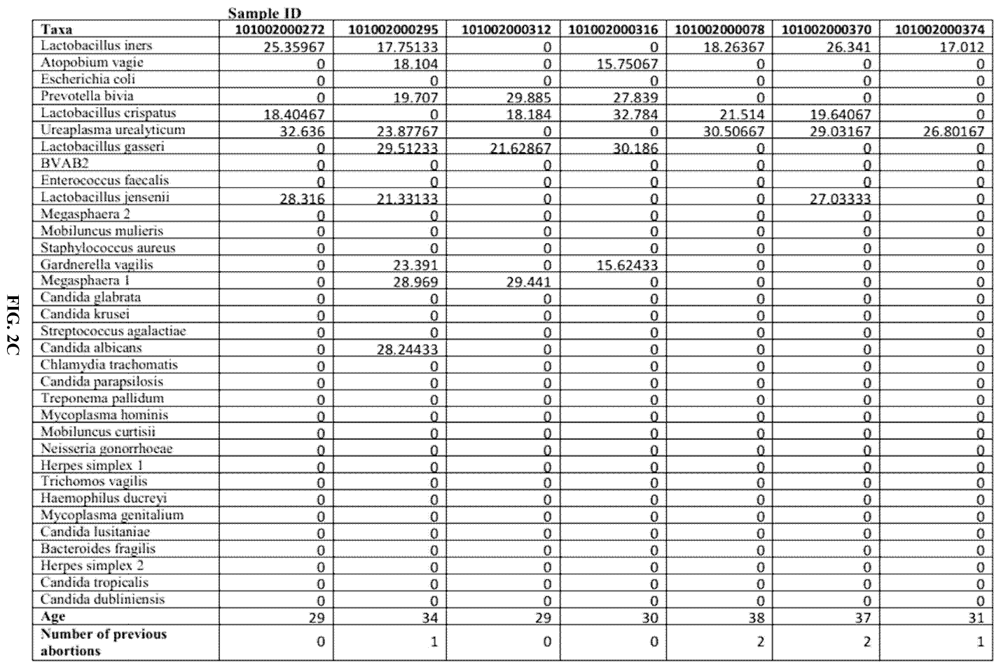

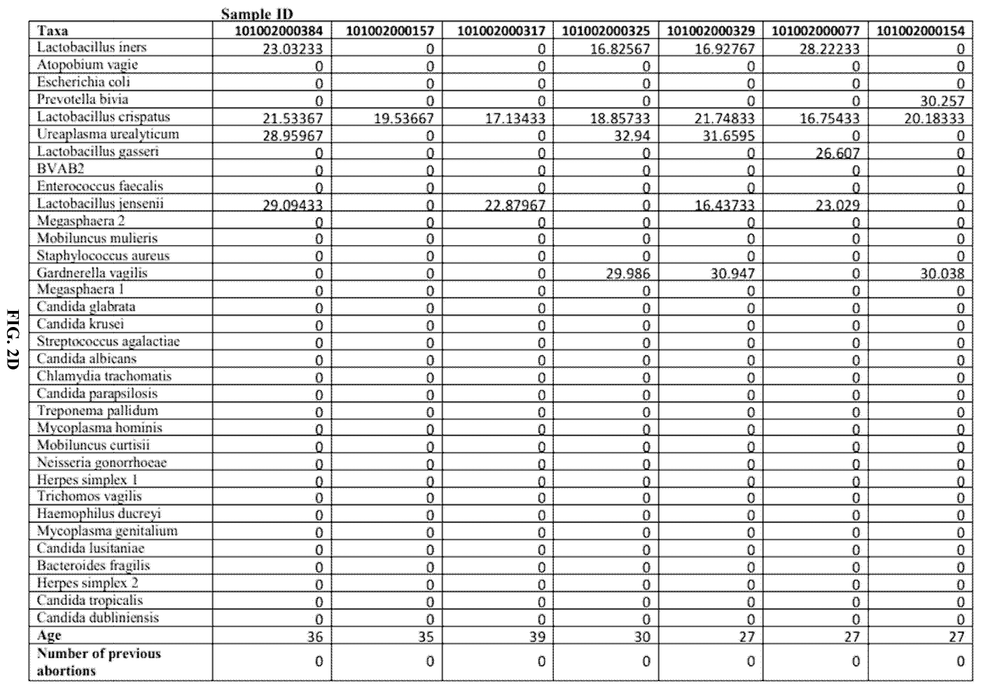

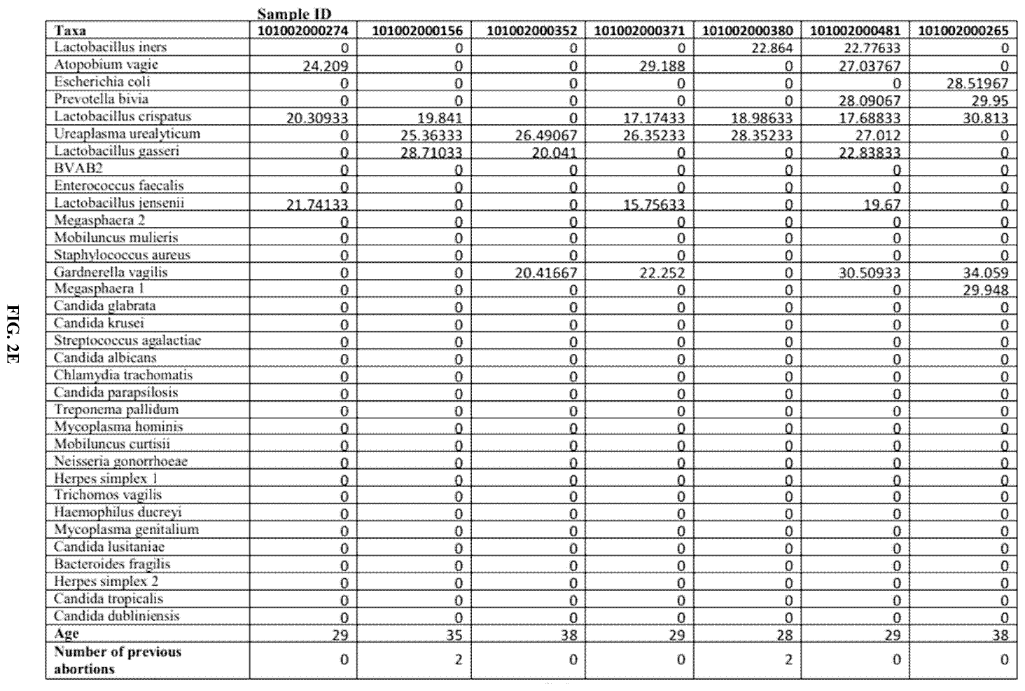

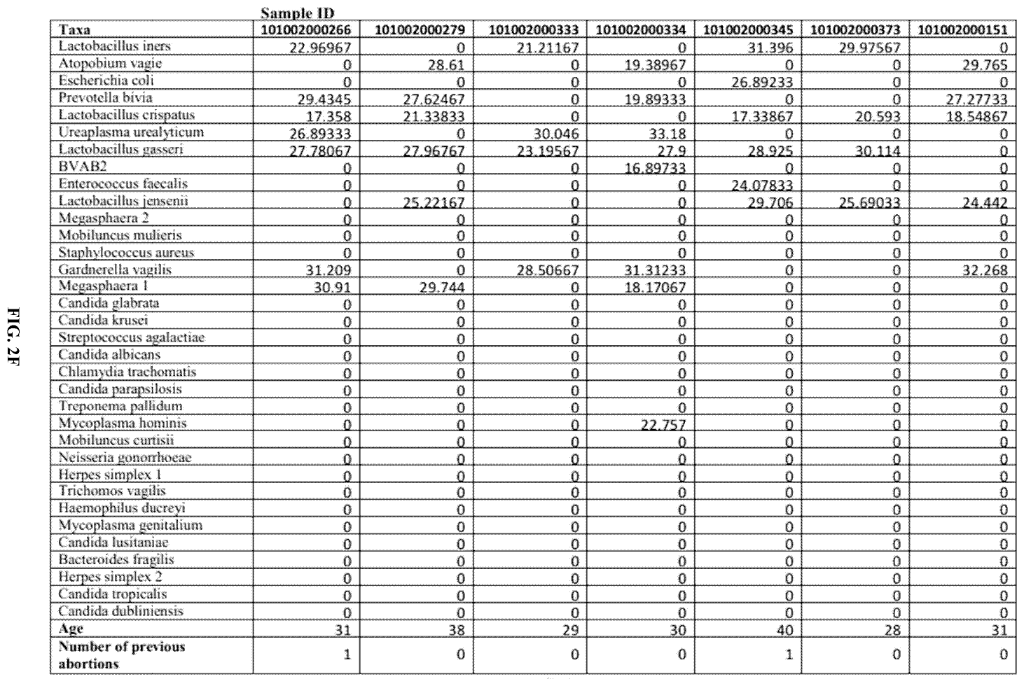

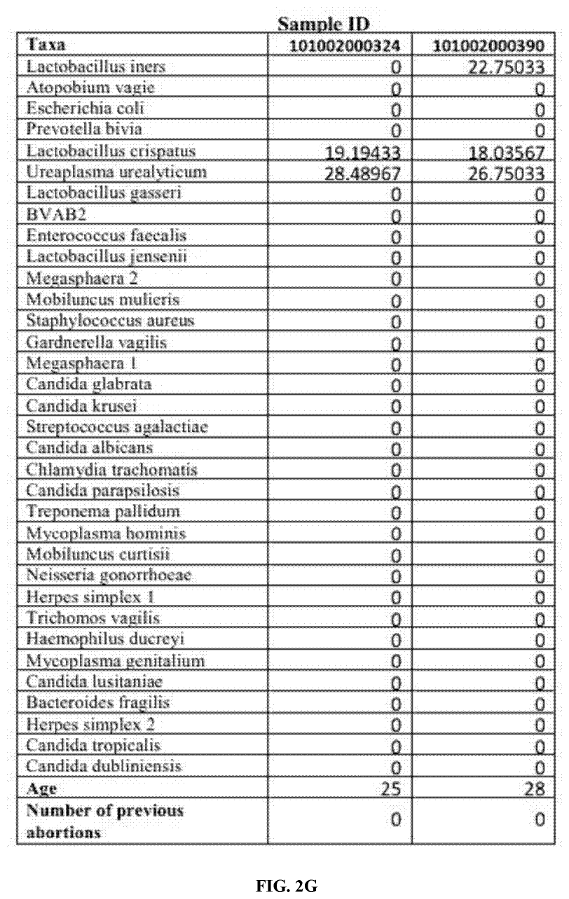

FIGS. 2A-2G illustrate an example of raw assay data showing the different amounts of 34 microbes found in each of the 44 test samples corresponding to Table 1 supra. In this example, the raw assay data shown in FIGS. 2A-2G provide the age of the subject, medical history of an abortion of the subject, and average Cᵣₜ values.

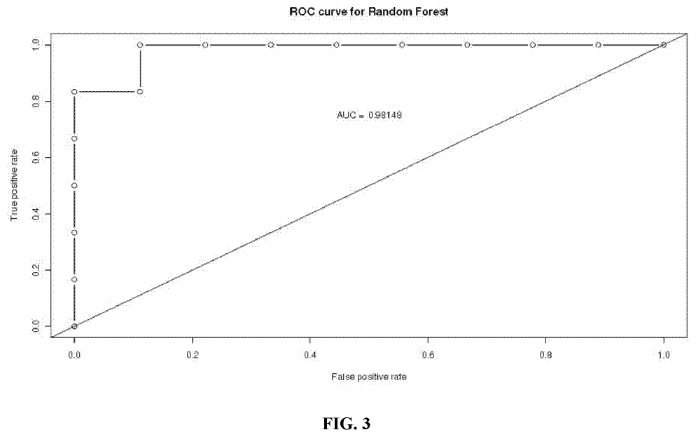

FIG. 3 illustrates an example of a Receiver Operator Characteristic (ROC) curve of a Random Forest (RF) classifier configured to predict premature birth condition based on analysis of microbe populations in vaginal samples, in accordance with some embodiments. In this example, the age of the subject, medical history of an abortion of the subject, and percentages of respective microbes were used as variables to train the algorithm.

The trained algorithm comprised a Random Forest classifier for predicting premature birth condition, which was trained by performing a plurality of successive runs. For each of the plurality of successive runs, a training partition was performed, in which at least 200, 250 or 300 biological samples were randomly selected as the training set (e.g., a set of independent training samples) for the Random Forest algorithm, and at least 20 biological samples (e.g., which was not previously selected for the training set) were designated as the testing set (e.g., a set of independent test samples). In an example, 44 biological samples were used as testing set.

The average performance metrics of this Random Forest classifier were:

Mean sensitivity ˜83.33%
Mean specificity ˜100.00%
Mean accuracy ˜93.33%
Mean precision ˜100.00%

### Mean F-Score ˜0.9091

Mean Area under ROC Curve (AUC) ˜0.9815

As further verification of the effectiveness of the Random Forest classifier, a blind-test data set were inputted into this trained Random Forest classifier, and a prediction accuracy of 93.33% was observed. In particular, after careful tuning of the probability cutoff value based on the F-Score curve (e.g., by adjusting the probability cutoff value to increase the F-Score value as close to 1 as possible), an even higher accuracy can be achieved for this blind-test data.

In an example, the blind-test data set can comprise 44 samples, and the age of the subject, medical history of an abortion of the subject, and percentages of respective microbes were used as variables to train the algorithm. The data of 44 test samples, including the predicted probability of premature birth condition (PBC) and predicted probability of normal birth (NORMAL) based on analysis of microbe populations in vaginal samples as well as actual birth result of each test sample, are shown in Table 2.

TABLE 2

| | Predicted | Predicted | | |
|---|---|---|---|---|
| | probability | probability | Predicted | Actual |
| Testing sample | of NORMAL | of Premature | birth | birth |
| ID | (Percentage) | Birth | result | result |
| 101002000481 | 11.6% | 88.4% | PROM | PROM |
| 101002000154 | 42.8% | 57.2% | PROM | PROM |
| 101002000274 | 42.6% | 57.4% | PROM | PROM |
| 101002000371 | 24.0% | 76.0% | PROM | PROM |
| 101002000077 | 37.2% | 62.8% | PROM | PROM |
| 101002000151 | 31.2% | 68.8% | PROM | PROM |
| 101002000156 | 34.0% | 66.0% | PROM | PROM |
| 101002000265 | 30.2% | 69.8% | PROM | PROM |
| 101002000324 | 38.4% | 61.6% | PROM | PROM |
| 101002000333 | 25.4% | 74.6% | PROM | PROM |
| 101002000345 | 34.6% | 65.4% | PROM | PROM |
| 101002000352 | 27.6% | 72.4% | PROM | PROM |
| 101002000380 | 38.0% | 62.0% | PROM | PROM |
| 101002000390 | 46.6% | 53.4% | PROM | PROM |
| 101002000334 | 58.2% | 41.8% | NORMAL | PROM |
| 101002000266 | 27.2% | 72.8% | PROM | PROM |
| 101002000279 | 48.0% | 52.0% | PROM | PROM |
| 101002000373 | 27.0% | 73.0% | PROM | PROM |
| 101002000075 | 82.4% | 17.6% | NORMAL | NORMAL |
| 101002000078 | 94.2% | 5.8% | NORMAL | NORMAL |
| 101002000106 | 89.8% | 10.2% | NORMAL | NORMAL |
| 101002000109 | 69.8% | 30.2% | NORMAL | NORMAL |
| 101002000128 | 95.0% | 5.0% | NORMAL | NORMAL |
| 101002000130 | 78.8% | 21.2% | NORMAL | NORMAL |
| 101002000138 | 59.0% | 41.0% | NORMAL | NORMAL |
| 101002000157 | 83.6% | 16.4% | NORMAL | NORMAL |
| 101002000163 | 65.6% | 34.4% | NORMAL | NORMAL |
| 101002000264 | 86.0% | 14.0% | NORMAL | NORMAL |
| 101002000270 | 64.6% | 35.4% | NORMAL | NORMAL |
| 101002000271 | 71.4% | 28.6% | NORMAL | NORMAL |
| 101002000272 | 63.8% | 36.2% | NORMAL | NORMAL |
| 101002000278 | 81.0% | 19.0% | NORMAL | NORMAL |
| 101002000286 | 97.4% | 2.6% | NORMAL | NORMAL |
| 101002000295 | 83.8% | 16.2% | NORMAL | NORMAL |
| 101002000312 | 65.4% | 34.6% | NORMAL | NORMAL |
| 101002000316 | 57.2% | 42.8% | NORMAL | NORMAL |
| 101002000317 | 81.8% | 18.2% | NORMAL | NORMAL |
| 101002000325 | 84.6% | 15.4% | NORMAL | NORMAL |
| 101002000329 | 75.4% | 24.6% | NORMAL | NORMAL |
| 101002000370 | 81.2% | 18.8% | NORMAL | NORMAL |
| 101002000374 | 90.6% | 9.4% | NORMAL | NORMAL |
| 101002000381 | 81.8% | 18.2% | NORMAL | NORMAL |
| 101002000384 | 78.0% | 22.0% | NORMAL | NORMAL |
| 101002000440 | 85.4% | 14.6% | NORMAL | NORMAL |

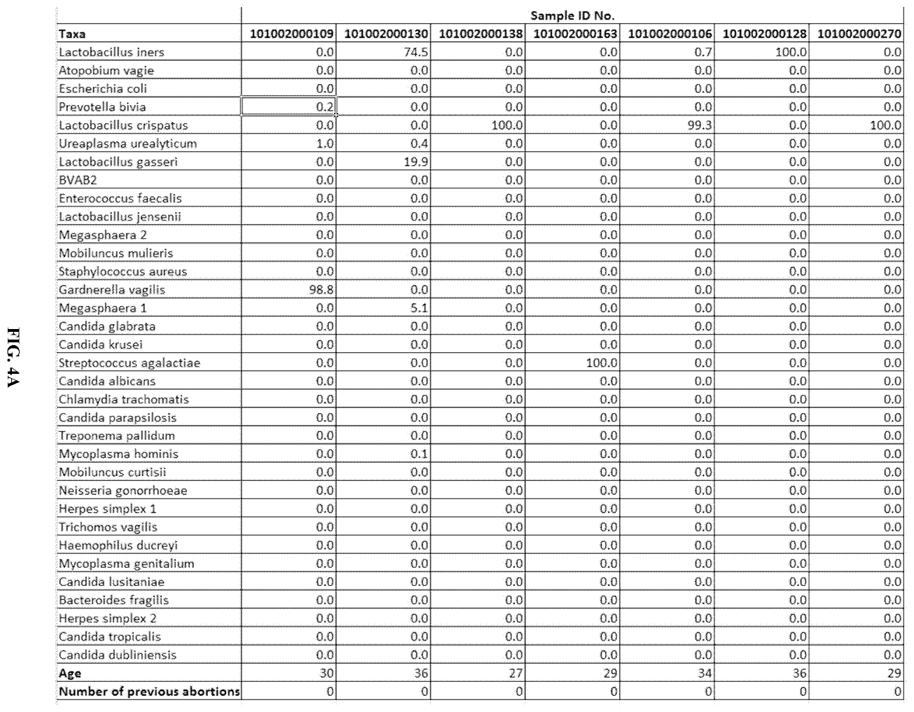

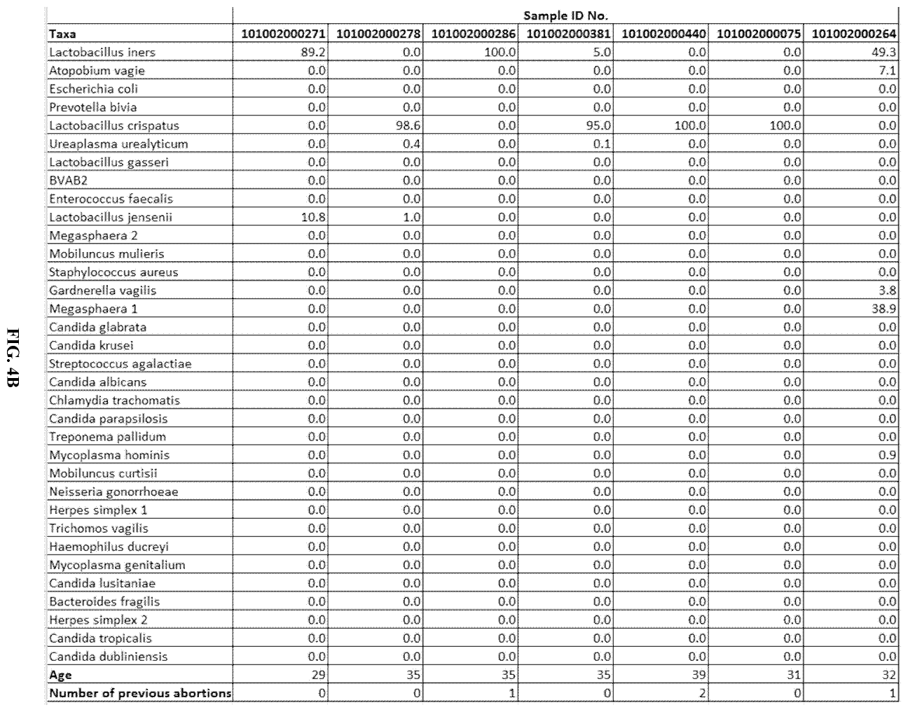

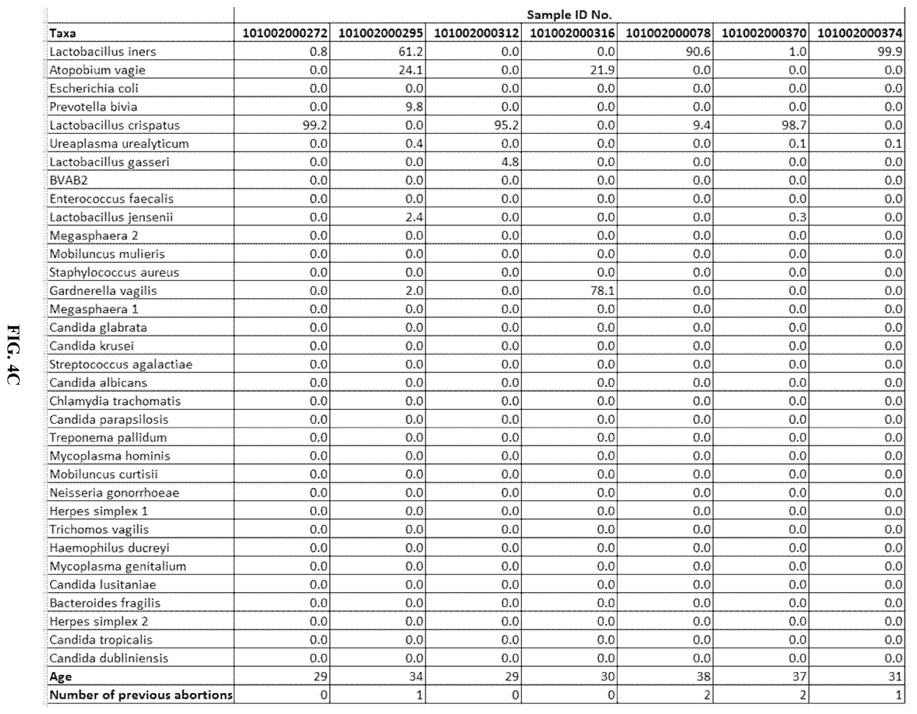

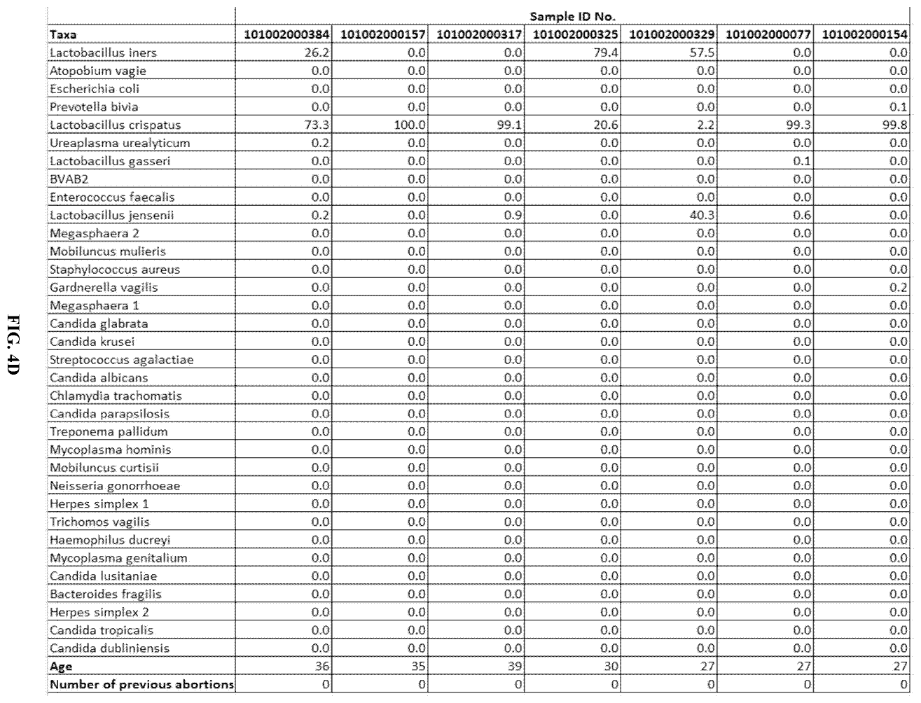

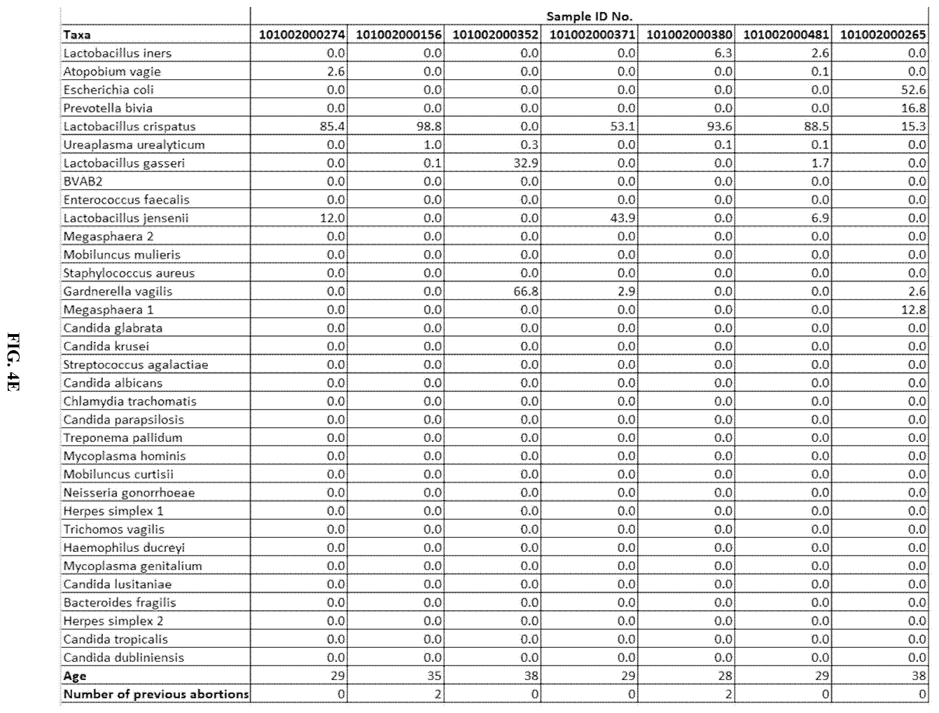

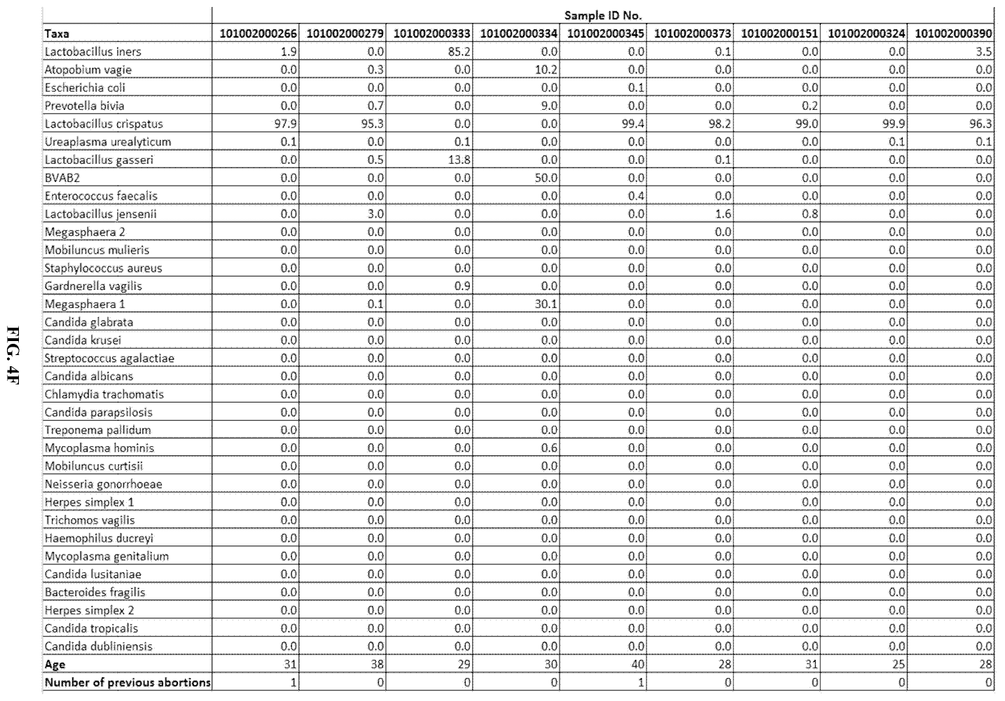

FIGS. 4A-4F illustrate an example of raw assay data showing the different amounts of 34 microbes found in each of the 44 test samples corresponding to Table 2 supra. In this example, the raw assay data shown in FIGS. 4A-4F provide the age of the subject, medical history of an abortion of the subject, and percentages of respective microbes.

### Predicting Premature Birth

After using a trained algorithm to process the data indicative of the distribution of the plurality of populations of microbes, the premature birth may be predicted in the subject with an accuracy of at least about 86.67%. The predicting may be based on the presence, absence, or relative amount of the individual population of the plurality of populations of microbes determined.

The premature birth may be predicted in the subject with an accuracy of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. The accuracy of predicting the premature birth by the trained algorithm may be calculated as the proportion of (1) independent test samples that are correctly predicted as having the premature birth and (2) independent test samples that are correctly predicted as not having the premature birth condition among all independent test samples.

The premature birth may be predicted in the subject with a positive predictive value (PPV) of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. The PPV of predicting the premature birth by the trained algorithm may be calculated as the proportion of independent test samples that are correctly predicted as having the premature birth among a sum of (1) independent test samples that are correctly predicted as having the premature birth and (2) independent test samples that are incorrectly predicted as having the premature birth. A PPV may also be referred to as a precision.

The premature birth may be predicted in the subject with a sensitivity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%. The sensitivity of predicting the premature birth by the trained algorithm may be calculated as the proportion of independent test samples that are correctly predicted as having the premature birth among a sum of (1) independent test samples that are correctly predicted as having the premature birth and (2) independent test samples that are incorrectly predicted as not having the premature birth.

The premature birth may be predicted in the subject with a clinical specificity of at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. The specificity of predicting the premature birth by the trained algorithm may be calculated as the proportion of independent test samples that are correctly predicted as not having the premature birth among a sum of (1) independent test samples that are correctly predicted as not having the premature birth and (2) independent test samples that are incorrectly predicted as having the premature birth.

The premature birth may be predicted in the subject with an F-score of at least about 0.05, at least about 0.10, at least about 0.15, at least about 0.20, at least about 0.25, at least about 0.30, at least about 0.35, at least about 0.40, at least about 0.50, at least about 0.65, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, or at least about 0.99. The F-score of predicting the premature birth by the trained algorithm may be calculated as the harmonic mean of the precision and the recall of the identification.

The method of predicting a premature birth can be performed to the subject more than one time during the pregnancy course. For example, the subject can be subject to the method at 10-12 weeks, 20-24 weeks and 28-32 weeks of pregnancy. Data indicative of a distribution of a plurality of populations of microbes of different types in the vaginal samples, which are sampled over time, can be compared to determine a change in likelihood of a premature birth in the patient and/or a progression or regression of the premature birth condition in the subject.

Upon predicting the subject as will have premature birth, the subject may be provided with a therapeutic intervention (e.g., prescribing an appropriate course of treatment to prevent the premature birth). The therapeutic intervention may comprise prescribing a contraction inhibitor, prescribing a magnesium sulfate, and prescribing a Glucocorticoid.

Microbiome distributions in a biological sample may be used to monitor a patient (e.g., a subject who is pregnant and at risk for premature birth condition). In such cases, the microbiome distribution of the patient may change during the course of treatment. For example, the microbiome distribution of a patient who is at risk for PROM may shift toward the microbiome distribution of a healthy subject (i.e., a subject that is not at risk for PROM). Conversely, for example, the microbiome distribution of a patient who is at risk for PROM may remain the same.

The progression or regression of the premature birth condition in the subject may be monitored by monitoring a course of treatment for treating the premature birth condition in the subject. The monitoring may comprise assessing the premature birth condition in the subject at two or more time points. The assessing may be based at least on the presence, absence, or relative amount of the individual populations of microbes of the plurality of populations of microbes determined at each of the two or more time points.

A difference in the presence, absence, or relative amount of the individual populations of microbes of the plurality of populations of microbes determined between the two or more time points may be indicative of one or more clinical indications, such as (i) a diagnosis of the premature birth condition in the subject, (ii) a prognosis of the premature birth condition in the subject, (iii) a progression of the premature birth condition in the subject, (iv) a regression of the premature birth condition in the subject, (v) an efficacy of the course of treatment for treating the premature birth condition in the subject, and (vi) a resistance of the premature birth condition toward the course of treatment for treating the premature birth condition in the subject.

A difference in the presence, absence, or relative amount of the individual populations of microbes of the plurality of populations of microbes determined between the two or more time points may be indicative of a diagnosis of the premature birth condition in the subject. For example, if the premature birth condition was not detected in the subject at an earlier time point but was detected in the subject at a later time point, then the difference is indicative of a diagnosis of the premature birth condition in the subject. A clinical action or decision may be made based on this indication of diagnosis of the premature birth condition in the subject, e.g., prescribing a new therapeutic intervention for the subject.

A difference in the presence, absence, or relative amount of the individual populations of microbes of the plurality of populations of microbes determined between the two or more time points may be indicative of a prognosis of the premature birth condition in the subject.

A difference in the presence, absence, or relative amount of the individual populations of microbes of the plurality of populations of microbes determined between the two or more time points may be indicative of a progression of the premature birth condition in the subject. For example, if the premature birth condition was detected in the subject both at an earlier time point and at a later time point, and if the difference is a negative difference (e.g., the presence, absence, or relative amount of the individual populations of microbes of the plurality of populations of microbes increased from the earlier time point to the later time point), then the difference may be indicative of a progression (e.g., increased tumor load, tumor burden, or tumor size) of the premature birth condition in the subject. A clinical action or decision may be made based on this indication of the progression, e.g., prescribing a new therapeutic intervention or switching therapeutic interventions (e.g., ending a current treatment and prescribing a new treatment) for the subject.

A difference in the presence, absence, or relative amount of the individual populations of microbes of the plurality of populations of microbes determined between the two or more time points may be indicative of a regression of the premature birth condition in the subject. For example, if the premature birth condition was detected in the subject both at an earlier time point and at a later time point, and if the difference is a positive difference (e.g., the presence, absence, or relative amount of the individual populations of microbes of the plurality of populations of microbes decreased from the earlier time point to the later time point), then the difference may be indicative of a regression (e.g., decreased tumor load, tumor burden, or tumor size) of the premature birth condition in the subject. A clinical action or decision may be made based on this indication of the regression, e.g., continuing or ending a current therapeutic intervention for the subject.

A difference in the presence, absence, or relative amount of the individual populations of microbes of the plurality of populations of microbes determined between the two or more time points may be indicative of an efficacy of the course of treatment for treating the premature birth condition in the subject. For example, if the premature birth condition was detected in the subject at an earlier time point but was not detected in the subject at a later time point, then the difference may be indicative of an efficacy of the course of treatment for treating the premature birth condition in the subject. A clinical action or decision may be made based on this indication of the efficacy of the course of treatment for treating the premature birth condition in the subject, e.g., continuing or ending a current therapeutic intervention for the subject.

A difference in the presence, absence, or relative amount of the individual populations of microbes of the plurality of populations of microbes determined between the two or more time points may be indicative of a resistance of the premature birth condition toward the course of treatment for treating the premature birth condition in the subject. For example, if the premature birth condition was detected in the subject both at an earlier time point and at a later time point, and if the difference is a negative or zero difference (e.g., the presence, absence, or relative amount of the individual populations of microbes of the plurality of populations of microbes increased or remained at a constant level from the earlier time point to the later time point), and if an efficacious treatment was indicated at an earlier time point, then the difference may be indicative of a resistance (e.g., increased or constant tumor load, tumor burden, or tumor size) of the course of treatment for treating the premature birth condition in the subject. A clinical action or decision may be made based on this indication of the resistance of the course of treatment for treating the premature birth condition in the subject, e.g., ending a current therapeutic intervention and/or switching to (e.g., prescribing) a different new therapeutic intervention for the subject.

### Outputting a Report of the Premature Birth Condition Prediction

After the premature birth condition is predicted in the subject, a report may be electronically outputted that indicates the risk or possibility of having premature birth condition. The report may be presented on a graphical user interface (GUI) of an electronic device of a user. The user may be the subject, a caretaker, a physician, a nurse, or another health care worker.

### Computer Control Systems

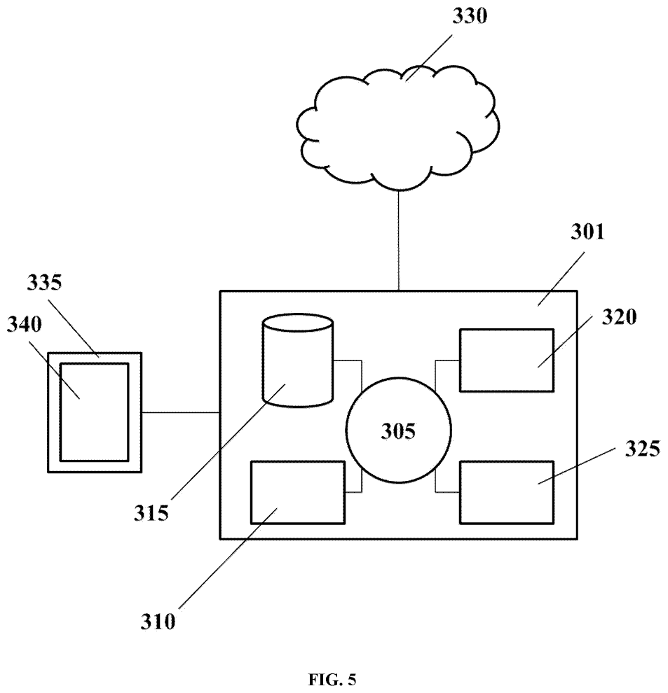

The present disclosure provides computer control systems that are programmed to implement methods of the disclosure. FIG. 5 shows a computer system 301 that is programmed or otherwise configured to, for example, (i) train and test a trained algorithm, (ii) use the trained algorithm to process data indicative of a distribution of a plurality of populations of microbes, (iii) determine a presence, absence, or relative amount of the individual populations of microbes of the plurality of populations of microbes in the biological sample, (iv) identify the subject as having the premature birth condition, or (v) electronically output a report that identifies or provides an indication of the progression or regression of the premature birth condition in the subject.

The computer system 301 can regulate various aspects of analysis, calculation, and generation of the present disclosure, such as, for example, (i) training and testing a trained algorithm, (ii) using the trained algorithm to process data indicative of a distribution of a plurality of populations of microbes, (iii) determining a presence, absence, or relative amount of the individual populations of microbes of the plurality of populations of microbes in the biological sample, (iv) identifying the subject as having the premature birth condition, or (v) electronically outputting a report that identifies or provides an indication of the progression or regression of the premature birth condition in the subject. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet.

The network 330 in some cases is a telecommunication and/or data network. The network 330 can include one or more computer servers, which can enable distributed computing, such as cloud computing. For example, one or more computer servers may enable cloud computing over the network 330 (“the cloud”) to perform various aspects of analysis, calculation, and generation of the present disclosure, such as, for example, (i) training and testing a trained algorithm, (ii) using the trained algorithm to process data indicative of a distribution of a plurality of populations of microbes, (iii) determining a presence, absence, or relative amount of the individual populations of microbes of the plurality of populations of microbes in the biological sample, (iv) identifying the subject as having the premature birth condition, or (v) electronically outputting a report that identifies or provides an indication of the progression or regression of the premature birth condition in the subject. Such cloud computing may be provided by cloud computing platforms such as, for example, Amazon Web Services (AWS), Microsoft Azure, Google Cloud Platform, and IBM cloud. The network 330, in some cases with the aid of the computer system 301, can implement a peer-to-peer network, which may enable devices coupled to the computer system 301 to behave as a client or a server.

The CPU 305 may comprise one or more computer processors and/or one or more graphics processing units (GPUs). The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 301 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, (i) a visual display indicative of training and testing of a trained algorithm, (ii) a visual display of data indicative of a distribution of a plurality of populations of microbes, (iii) a determined presence, absence, or relative amount of the individual populations of microbes of the plurality of populations of microbes in the biological sample, (iv) an identification of the subject as having the premature birth condition, or (v) an electronic report that identifies or provides an indication of the progression or regression of the premature birth condition in the subject. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, (i) train and test a trained algorithm, (ii) use the trained algorithm to process data indicative of a distribution of a plurality of populations of microbes, (iii) determine a presence, absence, or relative amount of the individual populations of microbes of the plurality of populations of microbes in the biological sample, (iv) identify the subject as having the premature birth condition, or (v) electronically output a report that identifies or provides an indication of the progression or regression of the premature birth condition in the subject.

### EXAMPLES

### Example 1—Prediction of Premature Birth Condition

In an example, a patient is 6 months pregnant and presents with the following risk factors: low socioeconomic status, history of past bleeding during her pregnancy, and a history of a premature birth in a previous pregnancy. A physician needs to identify the likelihood of a premature birth in the patient and recommends using the methods and systems provided herein to predict a likelihood of having a premature birth. A vaginal fluid sample from the patient is obtained in order to analyze the vaginal microbiome. The vaginal sample is processed in order to generate data indicative of a distribution of a plurality of populations of microbes of different types in the vaginal sample. A trained algorithm identifies the different types of microbes and identifies the presence, absence, or relative amount of individual populations of microbes, such as Lactobacillus, Escherichia, Prevotella, Enterococcus, Candida, Staphylococcus, and Herpes. The trained algorithm predicts the subject as having a risk of having a premature birth of about 88%. The trained algorithm predicts this risk percentage with an accuracy of 98.15%, based on the presence, absence, or relative amount of the individual populations of microbes in the vaginal sample. The system outputs an electronic report indicating there is an 88% risk of premature birth condition in the subject. The physician receives the electronic report and prescribes progesterone supplementation to the patient as a prophylactic measure against a premature birth condition occurring later in the pregnancy.

### Example 2—Prediction of Premature Birth Risks

In this example, the risk of premature birth in four pregnant women (i.e. Subject #1-4) showing signs for threat premature birth at different time points of pregnancy is evaluated by the present method. Specifically, the vaginal fluid sample from each of the subject is obtained and processed as shown in Example 1. The trained algorithm with an accuracy of 98.15% as shown in Example 1 is used to predict risk of premature birth condition in the subjects. The data of predicted probability of premature birth condition (PBC) and predicted birth result based on analysis of microbe populations in vaginal samples as well as actual birth result of each subject are shown in Table 3.

TABLE 3

| | | | Predicted | | |
|---|---|---|---|---|---|
| | | | probability | Predicted | Actual |
| Subject | Information for | | of premature | birth | birth |
| number | pregnancy | Microbes distribution | birth | result | result |
| 1 | Age: 37; | Lactobacillus crispatus: 99.73% | 82.0% | PROM | PROM at 33 |
| | Pregnant with twins; | Candida: 0.27%; | | | weeks of |
| | Show signs for threat | | | | pregnancy |
| | premature birth at 28 | | | | |
| | weeks of pregnancy | | | | |
| 2 | Age: 34 | Lactobacillus iners: 73.66% | 75.6% | PROM | PROM at 33 |
| | Pregnant with twins; | Gardnerella vagilis: 18.29% | | | weeks of |
| | Show signs for threat | Lactobacillus jensenii: 3.71% | | | pregnancy |
| | premature birth at 33 | Ureaplasma urealyticum: 1.52% | | | |
| | weeks of pregnancy | Candida: 1.37% | | | |
| | | BVAB2: 0.94% | | | |
| | | Atopobium vagie: 0.5% | | | |
| 3 | Age: 29; | Lactobacillus crispatus: 61.14% | 72.2% | PROM | PROM at 36 |
| | Show signs for threat | Gardnerella vagilis: 30.15% | | | weeks of |
| | premature birth at 36 | Lactobacillus iners: 6.89% | | | pregnancy |
| | weeks of pregnancy | Ureaplasma urealyticum: 1.56% | | | |
| | | Candida: 0.26% | | | |
| 4 | Age: 31; | Mycoplasma hominis: 36.67% | 97.3% | PROM | PROM at 21 |
| | A medical history of | Chlamydia trachomatis: 35.92% | | | weeks of |
| | abortion; | Ureaplasma urealyticum: 24.53% | | | pregnancy |
| | Show signs for threat | Candida: 2.88% | | | |
| | premature birth at 21 | | | | |
| | weeks of pregnancy | | | | |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for predicting premature birth condition in a subject having an unborn baby, comprising:
(a) processing a biological sample obtained from said subject to generate data indicative of a distribution of a plurality of populations of microbes of different types in said biological sample, wherein a presence, absence, or relative amount of an individual population of said plurality of populations of microbes is indicative of said premature birth condition in said subject;
(b) using a trained algorithm to process said data indicative of said distribution of said plurality of populations of microbes to determine a presence, absence, or relative amount of said individual population of said plurality of populations of microbes in said biological sample, which trained algorithm is configured to predict said premature birth condition at an accuracy of at least 90% for independent samples;
(c) based on said presence, absence, or relative amount of said individual population of said plurality of populations of microbes determined in (b), predicting said subject as having said premature birth condition in said subject at an accuracy of at least about 90%; and
(d) electronically outputting a report that identifies or provides an indication of said premature birth condition in said subject.

2. The method of claim 1, wherein said biological sample is independent of samples used to train said trained algorithm.

3. The method of claim 1, wherein said trained algorithm is configured to predict said premature birth condition with a negative predictive value (NPV) of at least about 90%.

4. The method of claim 3, wherein said NPV is at least about 95%.

5. The method of claim 1, wherein said trained algorithm is configured to predict said premature birth condition with a positive predictive value (PPV) of at least about 70%.

6. The method of claim 5, wherein said PPV is at least about 80%.

7. The method of claim 6, wherein said PPV is as at least about 90%.

8. The method of claim 7, wherein said PPV is as at least about 95%.

9. The method of claim 1, wherein said trained algorithm is configured to predict said premature birth condition with a clinical sensitivity of at least about 90%.

10. The method of claim 9, wherein said clinical sensitivity is at least about 95%.

11. The method of claim 10, wherein said clinical sensitivity at least about 99%.

12. The method of claim 1, wherein said trained algorithm is configured to predict said premature birth condition with an Area under Curve (AUC) of at least about 0.90.

13. The method of claim 12, wherein said AUC is at least about 0.95.

14. The method of claim 13, wherein said AUC is at least about 0.99.

15. The method of claim 1, wherein said subject does not display a premature birth condition.

16. The method of claim 1, wherein said biological sample is a vaginal fluid.

17. The method of claim 1, wherein said trained algorithm is trained with at least 200 independent training samples.

18. The method of claim 17, wherein said trained algorithm is trained with at least 250 independent training samples.

19. The method of claim 18, wherein said trained algorithm is trained with at least 300 independent training samples.

20. The method of claim 1, wherein said trained algorithm is trained with no more than 200 independent training samples associated with presence of a premature birth condition.

21. The method of claim 20, wherein said trained algorithm is trained with no more than 100 independent training samples associated with presence of said premature birth condition.

22. The method of claim 21, wherein said trained algorithm is trained with no more than 50 independent training samples associated with presence of said premature birth condition.

23. The method of claim 1, wherein said trained algorithm is trained with a first number of independent training samples associated with presence of a premature birth condition and a second number of independent training samples associated with absence of a premature birth condition, wherein the first number is no more than the second number.

24. The method of claim 1, wherein (a) comprises (i) subjecting said biological sample to conditions that are sufficient to isolate said plurality of populations of microbes, and (ii) identifying said presence, absence, or relative amount of said individual population of said plurality of populations of microbes.

25. The method of claim 24, further comprising extracting nucleic acid molecules from said biological sample, and subjecting said nucleic acid molecules to sequencing to identify said presence, absence, or relative amount of said individual population of said plurality of populations of microbes.

26. The method of claim 25, wherein said sequencing is massively parallel sequencing.

27. The method of claim 25, wherein said sequencing comprises nucleic acid amplification.

28. The method of claim 27, wherein said nucleic acid amplification is polymerase chain reaction (PCR).

29. The method of claim 25, wherein said sequencing comprises use of simultaneous reverse transcription (RT) and polymerase chain reaction (PCR).

30. The method of claim 25, further comprising using probes configured to selectively enrich nucleic acid molecules corresponding to said individual population of said plurality of populations of microbes.

31. The method of claim 30, wherein said probes are nucleic acid primers.

32. The method of claim 30, wherein said probes have sequence complementarity with nucleic acid sequences from said individual population of said plurality of populations of microbes.

33. The method of claim 1, wherein said plurality of populations of said plurality of populations of microbes comprise at least 5 different populations of microbes.

34. The method of claim 33, wherein said plurality of populations of said plurality of populations of microbes comprise at least 10 different populations of microbes.

35. The method of claim 33, wherein said at least 5 different populations microbes are different species of microbes.

36. The method of claim 35, wherein said at least 5 different species of microbes comprise one or more members selected from the group consisting of Lactobacillus iners, Atopobium vagie, Escherichia coli, Prevotella bivia, Lactobacillus crispatus, Ureaplasma urealyticum, Lactobacillus gasseri, BVAB2, Enterococcus faecalis, Lactobacillus jensenii, Megasphaera 2, Mobiluncus mulieris, Staphylococcus aureus, Gardnerella vagilis, Megasphaera 1, Candida glabrata, Candida krusei, Streptococcus agalactiae, Candida albicans, Chlamydia trachomatis, Candida parapsilosis, Treponema pallidum, Mycoplasma hominis, Mobiluncus curtisii, Neisseria gonorrhoeae, Herpes simplex 1, Trichomos vagilis, Haemophilus ducreyi, Mycoplasma genitalium, Candida lusitaniae, Bacteroides fragilis, Herpes simplex 2, Candida tropicalis, and Candida dubliniensis.

37. The method of claim 33, wherein said plurality of populations of microbes comprise one or more members selected from the group consisting of Lactobacillus gasseri, Gardnerella vagilis, Atopobium vagie, Ureaplasma urealyticum and Lactobacillus iners.

38. The method of claim 1, wherein said biological sample is processed to identify a distribution of a plurality of populations of microbes in said biological sample without any nucleic acid extraction.

39. The method of claim 1, wherein said report is presented on a graphical user interface of an electronic device of a user.

40. The method of claim 39, wherein said user is said subject.

41. The method of claim 1, wherein said premature birth condition is a preterm premature birth condition (PPROM).

42. The method of claim 41, wherein said premature birth condition causes chorioamnionitis, neonate sepsis, or both.

43. The method of claim 1, wherein said trained algorithm comprises a supervised machine learning algorithm.

44. The method of claim 43, wherein said supervised machine learning algorithm comprises a Random Forest, a support vector machine (SVM), a neural network, or a deep learning algorithm.

45. The method of claim 1, further comprising, upon predicting said subject as having said premature birth condition, providing said subject with a therapeutic intervention.

46. The method of claim 45, wherein said therapeutic intervention comprises recommending said subject for a secondary clinical test to confirm a diagnosis of said premature birth condition.

47. The method of claim 46, wherein said secondary clinical test comprises a blood test, an ultrasound scan, a fern test, an indigo carmine dye test, an immune-chromatological test, a nitrazine test, or a pooling test.

48. The method of claim 1, further comprising treating said subject upon predicting said subject as having said premature birth condition.

49. The method of claim 1, further comprising monitoring a course of treatment for treating a premature birth condition in said subject, wherein said monitoring comprises assessing said premature birth condition in said subject at two or more time points, wherein said assessing is based at least on said presence, absence, or relative amount of said individual population of said plurality of populations of microbes determined in (b) at each of said two or more time points.

50. The method of claim 49, wherein a difference in said presence, absence, or relative amount of said individual population of said plurality of populations of microbes determined in (b) between said two or more time points is indicative of one or more clinical indications selected from the group consisting of: (i) a diagnosis of said premature birth condition in said subject, (ii) a prognosis of said premature birth condition in said subject, (iii) a progression of said premature birth condition in said subject, (iv) a regression of said premature birth condition in said subject, (v) an efficacy of said course of treatment for treating said premature birth condition in said subject, and (vi) a resistance of said premature birth condition toward said course of treatment for treating said premature birth condition in said subject.

51. The method of claim 1, wherein said processing comprises assaying said biological sample using probes that are selected for said plurality of populations of microbes.

52. The method of claim 51, wherein said plurality of populations of microbes comprise at least 5 different populations of microbes.

53. The method of claim 52, wherein said plurality of populations of microbes comprise at least 10 different populations of microbes.

54. The method of claim 51, wherein said at least 5 different populations microbes are different species of microbes.

55. The method of claim 54, wherein said at least 5 different species of microbes comprise one or more members selected from the group consisting of Lactobacillus iners, Atopobium vagie, Escherichia coli, Prevotella bivia, Lactobacillus crispatus, Ureaplasma urealyticum, Lactobacillus gasseri, BVAB2, Enterococcus faecalis, Lactobacillus jensenii, Megasphaera 2, Mobiluncus mulieris, Staphylococcus aureus, Gardnerella vagilis, Megasphaera 1, Candida glabrata, Candida krusei, Streptococcus agalactiae, Candida albicans, Chlamydia trachomatis, Candida parapsilosis, Treponema pallidum, Mycoplasma hominis, Mobiluncus curtisii, Neisseria gonorrhoeae, Herpes simplex 1, Trichomos vagilis, Haemophilus ducreyi, Mycoplasma genitalium, Candida lusitaniae, Bacteroides fragilis, Herpes simplex 2, Candida tropicalis, and Candida dubliniensis.

56. The method of claim 51, wherein said plurality of populations of microbes comprise one or more members selected from the group consisting of Lactobacillus gasseri, Gardnerella vagilis, Atopobium vagie, Ureaplasma urealyticum and Lactobacillus iners.

57. The method of claim 51, wherein said probes are nucleic acid molecules having sequence complementarity with nucleic acid sequences of said plurality of populations of microbes.

58. The method of claim 57, wherein said nucleic acid molecules are primers or enrichment sequences.

59. The method of claim 51, wherein said assaying comprises use of array hybridization, polymerase chain reaction (PCR), or nucleic acid sequencing.

60. The method of claim 1, wherein said processing comprises assaying said biological sample using probes that are selective for said plurality of populations of microbes among other populations of microbes in said biological sample.

61. The method of claim 59, wherein said probes are nucleic acid molecules having sequence complementarity with nucleic acid sequences of said plurality of populations of microbes.

62. The method of claim 60, wherein said nucleic acid molecules are primers or enrichment sequences.

63. The method of claim 60, wherein said assaying comprises use of array hybridization, polymerase chain reaction (PCR), or nucleic acid sequencing.

64. A computer system for predicting a premature birth condition in a subject having an unborn baby, comprising:
a database that is configured to store data indicative of a distribution of a plurality of populations of microbes of different types in a biological sample of said subject, wherein a presence, absence, or relative amount of an individual population of said plurality of populations of microbes is indicative of said premature birth condition in said subject; and
one or more computer processors operatively coupled to said database, wherein said one or more computer processors are individually collectively programmed to:
(i) use a trained algorithm to process said data indicative of said distribution of said plurality of populations of microbes to determine a presence, absence, or relative amount of said individual population of said plurality of populations of microbes in said biological sample, which trained algorithm is configured to predict said premature birth condition at an accuracy of at least 90% for independent samples;
(ii) based on said presence, absence, or relative amount of said individual population of said plurality of populations of microbes determined in (b), predict said subject as having said premature birth condition in said subject at an accuracy of at least about 90%; and
(iii) electronically output a report that identifies or provides an indication of said premature birth condition in said subject.

65. The computer system of claim 64, further comprising an electronic display operatively coupled to said one or more computer processors, wherein said electronic display comprises a graphical user interface that is configured to display said report.

66. A computer control system programmed to implement the method of any of claims 1-63.

67. The computer control system of claim 66, wherein the computer control system is programmed to
(i) train and test a trained algorithm,
(ii) use the trained algorithm to process data indicative of a distribution of a plurality of populations of microbes,
(iii) determine a presence, absence, or relative amount of the individual populations of microbes of the plurality of populations of microbes in the biological sample,
(iv) identify the subject as having the premature birth condition, and optionally
(v) electronically output a report that identifies or provides an indication of the progression or regression of the premature birth condition in the subject.

68. A non-transitory computer readable medium comprising machine-executable code that, upon execution by one or more computer processors, implements a method for predicting premature birth condition in a subject having an unborn baby, said method comprising:
(a) process a biological sample obtained from said subject to generate data indicative of a distribution of a plurality of populations of microbes of different types in said biological sample, wherein a presence, absence, or relative amount of an individual population of said plurality of populations of microbes is indicative of said premature birth condition in said subject;
(b) using a trained algorithm to process said data indicative of said distribution of said plurality of populations of microbes to determine a presence, absence, or relative amount of said individual population of said plurality of populations of microbes in said biological sample, which trained algorithm is configured to predict said premature birth condition at an accuracy of at least 90% for independent samples;
(c) based on said presence, absence, or relative amount of said individual population of said plurality of populations of microbes determined in (b), predicting said subject as having said premature birth condition in said subject at an accuracy of at least about 90%; and
(d) electronically outputting a report that identifies or provides an indication of said premature birth condition in said subject.

69. A non-transitory computer readable medium comprising machine-executable code that, upon execution by one or more computer processors, implements the method of any of claims 1-63.

70. A kit for predicting premature birth in a subject having an unborn baby, comprising:
probes for identifying a presence, absence, or relative amount of individual populations of a plurality of populations of microbes of different types in a biological sample of said subject, wherein a presence, absence, or relative amount of said individual populations of said plurality of populations of microbes in said biological is indicative of a premature birth of said subject having said unborn baby, wherein said probes are selective for said plurality of populations of microbes among other populations of microbes in said biological sample; and
instructions for using said probes to process said biological sample to generate data indicative of a distribution of said plurality of populations of microbes of different types in said biological sample, to predict said premature birth at an accuracy of at least 90% for independent samples.

71. The kit of claim 70, wherein said probes are selective for said plurality of populations of microbes among other populations of microbes in said biological sample.

72. The kit of claim 71, wherein said plurality of populations of microbes comprise at least 5 different populations of microbes.

73. The kit of claim 72, wherein said plurality of populations of microbes comprise at least 10 different populations of microbes.

74. The kit of claim 71, wherein said at least 5 different populations microbes are different species of microbes.

75. The kit of claim 74, wherein said at least 5 different species of microbes comprise one or more members selected from the group consisting of Lactobacillus iners, Atopobium vagie, Escherichia coli, Prevotella bivia, Lactobacillus crispatus, Ureaplasma urealyticum, Lactobacillus gasseri, BVAB2, Enterococcus faecalis, Lactobacillus jensenii, Megasphaera 2, Mobiluncus mulieris, Staphylococcus aureus, Gardnerella vagilis, Megasphaera 1, Candida glabrata, Candida krusei, Streptococcus agalactiae, Candida albicans, Chlamydia trachomatis, Candida parapsilosis, Treponema pallidum, Mycoplasma hominis, Mobiluncus curtisii, Neisseria gonorrhoeae, Herpes simplex 1, Trichomos vagilis, Haemophilus ducreyi, Mycoplasma genitalium, Candida lusitaniae, Bacteroides fragilis, Herpes simplex 2, Candida tropicalis, and Candida dubliniensis.

76. The kit of claim 71, wherein said plurality of populations of microbes comprise one or more members selected from the group consisting of Lactobacillus gasseri, Gardnerella vagilis, Atopobium vagie, Ureaplasma urealyticum and Lactobacillus iners.

77. A kit for using in a method of any of claims 1-63, comprising:
probes for identifying a presence, absence, or relative amount of individual populations of a plurality of populations of microbes of different types in a biological sample of said subject, wherein a presence, absence, or relative amount of said individual populations of said plurality of populations of microbes in said biological is indicative of a premature birth of said subject having said unborn baby, wherein said probes are selective for said plurality of populations of microbes among other populations of microbes in said biological sample; and
instructions for using said probes to process said biological sample to generate data indicative of a distribution of said plurality of populations of microbes of different types in said biological sample, to predict said premature birth at an accuracy of at least 90% for independent samples.

78. Use of probes in the manufacture of a kit for the prediction of premature birth in a subject having an unborn baby,
wherein the probes is for identifying a presence, absence, or relative amount of individual populations of a plurality of populations of microbes of different types in a biological sample of said subject, wherein a presence, absence, or relative amount of said individual populations of said plurality of populations of microbes in said biological is indicative of a premature birth of said subject having said unborn baby, wherein said probes are selective for said plurality of populations of microbes among other populations of microbes in said biological sample, and
wherein the prediction comprises:
(a) processing a biological sample obtained from said subject to generate data indicative of a distribution of a plurality of populations of microbes of different types in said biological sample, wherein a presence, absence, or relative amount of an individual population of said plurality of populations of microbes is indicative of said premature birth condition in said subject;
(b) using a trained algorithm to process said data indicative of said distribution of said plurality of populations of microbes to determine a presence, absence, or relative amount of said individual population of said plurality of populations of microbes in said biological sample, which trained algorithm is configured to predict said premature birth condition at an accuracy of at least 90% for independent samples;
(c) based on said presence, absence, or relative amount of said individual population of said plurality of populations of microbes determined in (b), predicting said subject as having said premature birth condition in said subject at an accuracy of at least about 90%; and optionally
(d) electronically outputting a report that identifies or provides an indication of said premature birth condition in said subject.

79. The use of claim 78, wherein said probes are selective for said plurality of populations of microbes among other populations of microbes in said biological sample.

80. The use of claim 79, wherein said plurality of populations of microbes comprise at least 5 different populations of microbes.

81. The use of claim 80, wherein said plurality of populations of microbes comprise at least 10 different populations of microbes.

82. The use of claim 79, wherein said at least 5 different populations microbes are different species of microbes.

83. The use of claim 82, wherein said at least 5 different species of microbes comprise one or more members selected from the group consisting of Lactobacillus iners, Atopobium vagie, Escherichia coli, Prevotella bivia, Lactobacillus crispatus, Ureaplasma urealyticum, Lactobacillus gasseri, BVAB2, Enterococcus faecalis, Lactobacillus jensenii, Megasphaera 2, Mobiluncus mulieris, Staphylococcus aureus, Gardnerella vagilis, Megasphaera 1, Candida glabrata, Candida krusei, Streptococcus agalactiae, Candida albicans, Chlamydia trachomatis, Candida parapsilosis, Treponema pallidum, Mycoplasma hominis, Mobiluncus curtisii, Neisseria gonorrhoeae, Herpes simplex 1, Trichomos vagilis, Haemophilus ducreyi, Mycoplasma genitalium, Candida lusitaniae, Bacteroides fragilis, Herpes simplex 2, Candida tropicalis, and Candida dubliniensis.

84. The use of claim 79, wherein said plurality of populations of microbes comprise one or more members selected from the group consisting of Lactobacillus gasseri, Gardnerella vagilis, Atopobium vagie, Ureaplasma urealyticum and Lactobacillus iners.

85. Use of probes in the manufacture of a kit for the prediction of premature birth in a subject having an unborn baby,
wherein the probes identify a presence, absence, or relative amount of individual populations of a plurality of populations of microbes of different types in a biological sample of said subject, wherein a presence, absence, or relative amount of said individual populations of said plurality of populations of microbes in said biological is indicative of a premature birth of said subject having said unborn baby, wherein said probes are selective for said plurality of populations of microbes among other populations of microbes in said biological sample, and
wherein the kit is used in a method of any of claims 1-63.
